(19) United States
(12) Patent Application Publication
(10) Pub. No.: US 2014/0329833 A1
(43) Pub. Date: Nov. 6, 2014

(54) LYSINE DEMETHYLASE INHIBITORS FOR INFLAMMATORY DISEASES OR CONDITIONS

(71) Applicants: Tamara Maes, Castelldefels (ES); Marc Martinell Pedemonte, Barcelona (ES); Julio Castro-Palomino Laria, Barcelona (ES)

(72) Inventors: Tamara Maes, Castelldefels (ES); Marc Martinell Pedemonte, Barcelona (ES); Julio Castro-Palomino Laria, Barcelona (ES)

(73) Assignee: ORYZON GENOMICS, S.A, Cornella de Llobregat (ES)

(21) Appl. No.: 14/118,323

(22) PCT Filed: May 21, 2012
(86) PCT No.: PCT/EP2012/059377

§ 371 Date: Jun. 23, 2014

Related U.S. Application Data
(60) Provisional application No. 61/519,355, filed on May 19, 2011.

(51) Int. Cl. C07D 295/192; A61K 31/165; A61K 31/495; C07D 207/14; A61K 31/40; A61K 31/18; A61K 31/4418; C07C 215/64; A61K 31/135; C07C 311/08; A61K 45/06; C07C 237/06; C07D 213/38

(52) U.S. Cl. CPC C07D 295/192; C07C 237/06; A61K 31/165; A61K 31/495; C07D 207/14; A61K 31/40; C07D 213/38; A61K 31/4418; C07C 215/64; A61K 31/135; C07C 311/08; A61K 45/06; A61K 31/18
USPC 514/255.01; 514/619; 514/426; 514/357; 514/647; 514/605

(57) ABSTRACT

The invention relates to methods and compositions for the treatment or prevention of inflammation and inflammatory diseases or conditions. In particular, the invention relates to an LSD1 inhibitor, such as a 2-cyclylcylopropan-1-amine derivative, a phenelzine derivative and a propargylamine derivative, for use in treating or preventing inflammation and inflammatory diseases or conditions.

### TECHNICAL FIELD

The invention relates to methods and compositions for the treatment or prevention of inflammatory diseases. The invention also relates to an LSD1 inhibitor for use in treating or preventing inflammatory diseases or conditions.

### BACKGROUND

High platelet count can be caused by cancers, infections, splenectomy, anemia, and inflammatory diseases including rheumatoid arthritis and inflammatory bowel disease. A high platelet count can lead to excessive, dangerous blood clotting that can develop in deep vein thrombosis, stroke, or heart attack. Thrombosis and inflammatory diseases in humans are a major health problem. For example, atherothrombotic diseases and complications are the commonest cause of morbidity and mortality in developed countries. The role of platelets in both thrombosis and chronic inflammatory diseases such as atherosclerosis has been convincingly demonstrated (e.g. D. Wagner et al. (2003) Arteriosclerosis, Thrombosis, and Vascular Biology 23:2131-2137).

For many years it has been known that platelets play an important role in thrombosis and homeostasis. Platelet adhesion as well as platelet recruitment and aggregation are implicated in thrombus formation. Rinder H M (et al. (1998) Blood, 91(4):1288-1294), evaluated platelet kinetics to show that increased percentages and absolute numbers of reticulated platelets (RP) are highly associated with thrombosis in patients with thrombocytosis.

In recent times, however, it has become evident that platelets also have relevant functions in inflammation. It was shown that thrombosis and inflammation share several key molecular mechanisms and in fact are two intrinsically linked processes (Wagner D. et al. Arteriosclerosis, Thrombosis, and Vascular Biology 2003; 23:2131-2137). The release of platelet granular contents, including adhesive proteins, growth factors and chemokinescytokines, that serve to facilitate hemostasis and wound repair, also function in acute and chronic inflammatory disease. Moreover, platelets play a vital role in the recruitment of leukocytes into inflamed tissue.

Mannaioni P. F. (et al. (1997) Inflamm. Res. 46(1):4-18) discussed the participation of platelets in overtly inflammatory disorders, such as acute respiratory distress syndrome, mesangial glomerulonephritis, chronic inflammatory bowel disease (IBD), disseminated intravascular inflammation, and allergic vasculitis. The physiologic reference range of platelet counts is 150-400×10⁹/L. A platelet count exceeding the upper limit is common in patients with inflammatory bowel disease; the increase in the platelet count is a reactive phenomenon to the inflammatory process. Moreover, several lines of evidence support a role for platelets in ulcerative colitis and Crohn's disease, the two most common forms of IBD (Danese, S. et al. (2005) Crit. Rev. Immunol., 25:103-121). Danese et al (Am J Gastroenterol, 2004, 99(5):938-45) have reported that platelets are on the way to acquire a higher degree of relevance in the complex mosaic of inflammatory bowel disease pathogenesis. Because both their number and state of activation are markedly increased during the active and even inactive stage of Crohn's disease or ulcerative colitis, their presence represents a significant risk factor for amplification of gut inflammation and makes them a rational target for specific therapeutic intervention.

Besides their role in hemostasis and thrombosis, platelets regulate a variety of other inflammatory responses and are key players in atherothrombosis. Platelet-induced chronic inflammatory processes at the vascular wall result in development of atherosclerotic lesions and atherothrombosis (Mein ad Gawaz et al. (2005) J. Clin Invest. 115(12):3378-3384). Furthermore, it is well-documented that platelets accumulate in the synovial fluid of individuals with rheumatoid arthritis, E. Bollard (et al. Science 2010, Vol. 327 no. 5965, 580-583) investigated the role of platelets in the autoimmune disease rheumatoid arthritis and demonstrated platelet participation in inflammatory arthritis in vivo. Platelets have also been suggested to play a role in the pathomechanism of chronic skin inflammatory diseases such as atopic dermatitis and psoriasis (R Tamagawa-Mineoka et al, 2008, Allergology International, 57:391-396).

A role for platelets in various pulmonary conditions has been postulated. In asthma, platelets have been found to actively participate in most of its main features, including bronchial hyperresponsiveness, bronchoconstriction, airway inflammation and airway remodeling (K N Kornerup et al, (2007), Platelets, 18(5), 319-28). G. Cakmak et al. (2009) Int. J. Med. Med. Sci. 1(5):227-229 first reported that platelet number is an indicator of systemic inflammation in patients with Chronic obstructive pulmonary disease (COPD). Biljak V R et al (Platelets, 2011, 22(6) 466-70, epub 2011 Apr. 20) reported that patients with COPD have a significantly increased platelet count along with a reduced platelet volume when compared to healthy controls. J D Maclay (et al, Thorax, 2011, 66(9), 769-74, epub 2011 Apr. 20) reported increased platelet activation in patients with stable and acute exacerbation of COPD; according to the author, these findings identify a novel mechanism to explain the increased cardiovascular risk in COPD and suggest platelet inhibition as a plausible therapeutic target. The platelets of individuals with asthma have higher than control levels of expressed P-selectin (C. Moritani, et al. (1998) Chest, 113:452-458), and platelet-derived P-selectin functions to recruit leukocytes to their lungs (Pitchford et al. (2005) Blood, 105:2074-2081). The evidence points toward platelets being an attractive new target in the area of asthma research; a target with much-needed novel therapeutic potential (K N Kornerup et al, (2007), Platelets, 18(5), 319-28). O'Sullivan et al ((2006) Am J Respir Crit Care Med, vol 173, 483-90) reviewed the inflammatory role of platelets in cystic fibrosis (CF) and hypothesized that platelets are a potential therapeutic target in patients with CF.

A group of enzymes known as lysine methyl transferases and lysine demethylases are involved in histone lysine modifications. One particular human lysine demethylase enzyme called Lysine Specific Demethylase-1 (LSD1) was recently discovered (Shi et al., (2004) Cell 119:941) and shown to be involved in histone lysine methylation. LSD1 has a fair degree of structural similarity, and amino acid identityhomology to polyamine oxidases and monoamine oxidases, all of which (i.e., MAO-A, MAO-B and LSD1) are flavin dependent amine oxidases which catalyze the oxidation of nitrogen-hydrogen bonds and/or nitrogen-carbon bonds. Although the main target of LSD1 appears to be mono- and di-methylated histone lysines, specifically H3K4 and H3K9, there is evidence in the literature that LSD1 can demethylate methylated lysines on non-histone proteins like p53, E2F1, Dnmt1 and STAT3.

Several groups have reported LSD1 inhibitors in the literature. Sharma et al. recently reported a new series of urea and thiourea analogs based on an earlier series of polyamines which were shown to inhibit LSD1 and modulate histone methylation and gene expression in cells (Sharma et al (2010) J. Med. Chem. 22; 53 (14):5197-212). Some efforts were made to make analogs of the histone peptide that is methylated by the enzyme, other efforts have focused on more small molecule like molecules based on known MAO inhibitors. Gooden et al. reported trans-2-arylcyclopropylamine analogues that inhibit LSD1 with Ki values in the range of 188-566 micromolar (Gooden et al. (2008) Bioorg. Med. Chem. Let. 18:3047-3051). Most of these compounds were more potent against MAO-A as compared to MAO-B. Ueda (et al. (2009) J. Am. Chem. Soc. 131(48):17536-17537) reported cyclopropylamine analogs selective for LSD1 over MAO-A and MAO-B that were designed based on reported X-ray crystal structures of these enzymes with a phenylcyclopropylamine-FAD adduct and a FAD-N-propargyl lysine peptide. The reported IC50 values for phenylcyclopropylamine were about 32 micromolar for LSD1 whereas compounds 1 and 2 had values of 2.5 and 1.9 micromolar respectively.

Importantly, studies have also been conducted on amine oxidase inhibitor compounds to determine selectivity for MAO-A versus MAO-B since MAO-A inhibitors can cause dangerous side-effects (see, e.g., Yoshida et. al. (2004) Bioorg. Med. Chem. 12(10):2645-2652, Hruschka et al. (2008) Biorg. Med. Chem. (16):7148-7166, Folks et al. (1983) J. Clin. Psychopharmacol. (3):249 and Youdim et al. (1983) Mod. Probl. Pharmacopsychiatry (19):63).

The current platelet research focuses on the development of new anti-platelet drugs and has strong support from various drug companies. So far, simultaneous use of different anti-platelet drugs that are directed against different targets has been effective in reducing adverse clinical events. Anti-platelet drugs play a well-defined role in the primary and secondary prevention of arterial thrombotic disorders. Furthermore, anti-platelet therapy is effective in decreasing the incidence of serious non-fatal and fatal complications in patients with symptomatic atherothrombotic diseases. This is a prevalent disease and its complications are the commonest cause of morbidity and mortality in the elderly. The multiple effects of platelets in inflammatory diseases suggest that anti-platelet therapy will produce clinical benefit in these disorders (Archibald McNicol et al. (2008) Cardiovascular & Haematological Disorders-Drug Targets, 8:99-117). Therefore, the anti-inflammatory effects associated with the anti-platelet therapy may contribute in part to the clinical beneficial effects of new drugs.

There is a need for new drugs for inflammatory diseases that target novel points of intervention in the disease processes and avoid side-effects associated with certain targets.

### SUMMARY OF THE INVENTION

The present invention relates to the treatment or prevention of inflammatory diseases or conditions. The inventors have unexpectedly found that inhibitors of LSD1 reduce platelets and can therefore be used for the treatment or prevention of inflammatory diseases or conditions. This finding was particularly unexpected since LSD1 inhibition was shown to have a specific effect of reducing platelets in animal studies. Advantageously, the use of selective LSD1 inhibitors or dual LSD1/MAO-B inhibitors avoids side-effects associated with targets such as MAO-A. The inventors found that administration of LSD1 inhibitors chronically was well tolerated in mammals (selective and dual LSD1/MAO-B inhibitors). Thus, the inventors have unexpectedly found that LSD1 inhibition, selective LSD1 inhibition or LSD1/MAO-B dual inhibition represent a new therapeutic approach to treating or preventing inflammatory diseases or conditions.

The present invention provides for the treatment or prevention of inflammation or an inflammatory disease or condition. In particular, the invention provides compositions and methods that can be used to reduce platelets or other blood cells and medical benefits derived therefrom.

Thus, according to the invention, the treatment or prevention of inflammation, an inflammatory disease or condition, and in particular when caused by or associated with an increased platelet count in an individual, comprises administering to an individual in need of treatment or prevention, a therapeutically effective amount of a LSD1 inhibitor. The individual in need of treatment or prevention can be a human or, e.g., another mammal. In one aspect, the therapeutically effective amount is an amount sufficient to reduce platelets.

Accordingly, the invention provides for the treatment or prevention of inflammation or an inflammatory disease or condition using methods and compositions based on modulators, particularly inhibitors, of LSD1. The invention thus relates to an LSD1 inhibitor for use in the treatment or prevention of inflammation or an inflammatory disease or condition. The invention also relates to a pharmaceutical composition comprising an LSD1 inhibitor and a pharmaceutically acceptable carrier for use in the treatment or prevention of inflammation or an inflammatory disease or condition. The invention further relates to an LSD1 inhibitor, or a pharmaceutical composition comprising an LSD1 inhibitor and a pharmaceutically acceptable carrier, for use in the treatment or prevention of inflammation or an inflammatory disease or condition by reducing platelet levels.

The inflammation or inflammatory disease or condition to be treated or prevented in accordance with the present invention includes, without being limited thereto: atherosclerosis, a respiratory inflammatory disorder (e.g. respiratory distress syndrome, asthma, COPD, bronchial hyperresponsiveness, bronchoconstriction, airway inflammation, airway remodeling or cystic fibrosis), chronic inflammatory bowel disease, ulcerative colitis, Crohn's disease, a chronic skin inflammatory disease (e.g. psoriasis or atopic dermatitis), mesangial glomerulonephritis, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, hepatic cirrhosis, or nephritis. In particular, the inflammation or inflammatory disease or condition to be treated or prevented in accordance with the invention includes: atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis. In the context of the present invention, the compounds and pharmaceutical compositions according to the invention, including LSD1 inhibitors and the specific compounds described herein, are envisaged to be used particularly in the treatment or prevention of inflammation or an inflammatory disease or condition selected from atherosclerosis, respiratory distress syndrome, mesangial glomerulonephritis, chronic inflammatory bowel disease, ulcerative colitis, Crohn's disease, disseminated intravascular inflammation, allergic vasculitis, arthritis, rheumatoid arthritis, asthma, COPD, bronchial hyperresponsiveness, bronchoconstriction, airway inflammation, airway remodeling, cystic fibrosis, atopic dermatitis or psoriasis.

In one embodiment, the invention provides a method of treating or preventing inflammation or an inflammatory disease or condition, in an individual by administering a therapeutically effective amount of a LSD1 inhibitor to the individual. In one particular embodiment, said inflammation or inflammatory disease or condition is atherosclerosis, a respiratory inflammatory disorder (e.g. respiratory distress syndrome, asthma, COPD, bronchial hyperresponsiveness, bronchoconstriction, airway inflammation, airway remodeling or cystic fibrosis), chronic inflammatory bowel disease, ulcerative colitis, Crohn's disease, a chronic skin inflammatory disease (e.g. psoriasis or atopic dermatitis), mesangial glomerulonephritis, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, hepatic cirrhosis, or nephritis. In one particular embodiment, said inflammation or inflammatory disease or condition is atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis. In one particular embodiment, said inflammation or inflammatory disease or condition is atherosclerosis, respiratory distress syndrome, mesangial glomerulonephritis, chronic inflammatory bowel disease, ulcerative colitis, Crohn's disease, disseminated intravascular inflammation, allergic vasculitis, arthritis, rheumatoid arthritis, asthma, COPD, bronchial hyperresponsiveness, bronchoconstriction, airway inflammation, airway remodeling, cystic fibrosis, atopic dermatitis or psoriasis. In one particular embodiment, said inflammation or inflammatory disease or condition is atherosclerosis. In one particular embodiment, said inflammation or inflammatory disease or condition is a respiratory inflammatory disorder, such as respiratory distress syndrome, asthma, COPD, bronchial hyperresponsiveness, bronchoconstriction, airway inflammation, airway remodeling or cystic fibrosis. In one particular embodiment, said inflammation or inflammatory disease or condition is chronic inflammatory bowel disease, such as ulcerative colitis or Crohn's disease. According to one aspect of this embodiment, the LSD1 inhibitor is a small molecule. According to one aspect of this embodiment, the LSD1 inhibitor is an irreversible or a reversible amine oxidase inhibitor. In one aspect, the amine oxidase inhibitor is a phenylcyclopropylamine derivative or analog (for example an arylcyclopropylamine derivative or a heteroarylcyclopropylamine derivative), a phenelzine derivative or analog, or a propargylamine derivative or analog. In one aspect, the LSD1 inhibitor is an arylcyclopropylamine derivative or a heteroarylcyclopropylamine derivative.

In another embodiment, the invention provides a method of treating or preventing inflammation or an inflammatory disease or condition, in an individual by administering a therapeutically effective amount of a LSD1 inhibitor wherein the therapeutically effect amount is an amount sufficient to reduce platelets. According to one aspect of this embodiment, the LSD1 inhibitor is a small molecule. According to one aspect of this embodiment, the LSD1 inhibitor is an irreversible or a reversible amine oxidase inhibitor. In one aspect, the amine oxidase inhibitor is a phenylcyclopropylamine derivative or analog (for example an arylcyclopropylamine derivative or a heteroarylcyclopropylamine derivative), a phenelzine derivative or analog, or a propargylamine derivative or analog. In one aspect, the LSD1 inhibitor is an arylcyclopropylamine derivative or a heteroarylcyclopropylamine derivative. In one particular embodiment, said inflammation or inflammatory disease or condition is atherosclerosis, respiratory inflammatory disorders (e.g. respiratory distress syndrome, asthma, COPD, bronchial hyperresponsiveness, bronchoconstriction, airway inflammation, airway remodeling and cystic fibrosis), mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, hepatic cirrhosis, nephritis or chronic skin inflammatory diseases (e.g. psoriasis and atopic dermatitis). In another particular embodiment, said inflammation or inflammatory disease or condition is atherosclerosis, respiratory distress syndrome, mesangial glomerulonephritis, chronic inflammatory bowel disease, ulcerative colitis, Crohn's disease, disseminated intravascular inflammation, allergic vasculitis, arthritis, rheumatoid arthritis, asthma, COPD, bronchial hyperresponsiveness, bronchoconstriction, airway inflammation, airway remodeling, cystic fibrosis, atopic dermatitis or psoriasis.

The invention further provides a method of identifying compounds that have activity against inflammatory diseases or conditions. More particularly, the method involves identifying a compound that inhibits LSD1 and then testing the LSD1 inhibitors in an assay for inflammation or an inflammatory disease or condition. According to this embodiment an assay system is employed to detect compounds and/or compositions that affect inflammation or an inflammatory disease or condition.

The invention, in one embodiment, is a method of treating or preventing a symptom of inflammation or an inflammatory disease or condition, comprising identifying a patient in need of such treatment or prevention and administering to the individual an amount of a LSD1 inhibitor sufficient to improve the symptom or reduce the rate of decline (i.e. worsening) of the symptom, thereby treating or preventing the symptom. One such symptom is excessive or elevated platelet or other blood cell levels, particularly excessive or elevated platelet levels. In a related aspect, the invention is the use of a LSD1 inhibitor in an amount sufficient to modulate LSD1 activity for treating or preventing an inflammatory disease or condition, in an individual having one of these diseases or conditions. In a related aspect, the invention is the use of a LSD1 inhibitor in an amount sufficient to modulate LSD1 activity for treating or preventing atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis, in an individual having any of these diseases. In a related aspect, the invention is the use of a LSD1 inhibitor in an amount sufficient to modulate LSD1 activity for treating or preventing atherosclerosis, respiratory distress syndrome, mesangial glomerulonephritis, chronic inflammatory bowel disease, ulcerative colitis, Crohn's disease, disseminated intravascular inflammation, allergic vasculitis, arthritis, rheumatoid arthritis, asthma, COPD, bronchial hyperresponsiveness, bronchoconstriction, airway inflammation, airway remodeling, cystic fibrosis, atopic dermatitis or psoriasis, in an individual having any of these diseases. In one embodiment of this aspect, the amount of LSD1 inhibitor administered is sufficient to modulate or inhibit LSD1 activity while not substantially inhibiting MAO-A activity, thereby avoiding or reducing side-effects associated with administration of MAO-A inhibitors.

In one aspect, the invention relates to a pharmaceutical composition comprising a therapeutically effective amount of a LSD1 inhibitor and a pharmaceutically acceptable carrier for use in treating or preventing inflammation or an inflammatory disease or condition. In one aspect, a therapeutically effective amount of the composition is administered to an individual in an amount sufficient to prevent or treat said disease or condition. In another aspect, a therapeutically effective amount of the composition is administered to an individual in an amount sufficient to reduce platelets, and particularly reduce the platelet count in the individual. In another aspect, the amount of LSD1 inhibitor administered is sufficient to modulate or inhibit LSD1 activity. In another aspect, the inflammatory disease or condition is atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis. In another aspect, said inflammation or inflammatory disease or condition is atherosclerosis, respiratory distress syndrome, mesangial glomerulonephritis, chronic inflammatory bowel disease, ulcerative colitis, Crohn's disease, disseminated intravascular inflammation, allergic vasculitis, arthritis, rheumatoid arthritis, asthma, COPD, bronchial hyperresponsiveness, bronchoconstriction, airway inflammation, airway remodeling, cystic fibrosis, atopic dermatitis or psoriasis.

In one aspect, the invention relates to a pharmaceutical composition for treating inflammation or an inflammatory disease or condition, or a related disease or condition comprising a platelet reducing effective amount of a LSD1 inhibitor. In one embodiment of this aspect, the inflammatory disease or condition is atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis. In another embodiment of this aspect, said inflammation or inflammatory disease or condition is atherosclerosis, respiratory distress syndrome, mesangial glomerulonephritis, chronic inflammatory bowel disease, ulcerative colitis, Crohn's disease, disseminated intravascular inflammation, allergic vasculitis, arthritis, rheumatoid arthritis, asthma, COPD, bronchial hyperresponsiveness, bronchoconstriction, airway inflammation, airway remodeling, cystic fibrosis, atopic dermatitis or psoriasis.

In one aspect, the invention relates to a pharmaceutical composition for treating inflammation or an inflammatory disease or condition, wherein the pharmaceutical composition comprises a platelet reducing effective amount of a LSD1 inhibitor and a pharmaceutically acceptable carrier. In one embodiment of this aspect, the inflammatory disease or condition is atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis. In another embodiment, said inflammation or inflammatory disease or condition is atherosclerosis, respiratory distress syndrome, mesangial glomerulonephritis, chronic inflammatory bowel disease, ulcerative colitis, Crohn's disease, disseminated intravascular inflammation, allergic vasculitis, arthritis, rheumatoid arthritis, asthma, COPD, bronchial hyperresponsiveness, bronchoconstriction, airway inflammation, airway remodeling, cystic fibrosis, atopic dermatitis or psoriasis.

In one aspect, the invention relates to a method of combination treatment. According to this method a LSD1 inhibitor and a second agent, which is an anti-platelet agent are administered to an individual (e.g. a human) in need of treatment wherein the individual has an inflammation or inflammatory disease or condition. In a more specific aspect, said anti-platelet agent is chosen from Aspirin, Clopidogrel, Prasugrel, Ticlopidine, Cilostazol, Abciximab, Eptifibatide, Tirofiban, Dipyridamole, Anagrelide, Hydroxyurea, or Epoprostenol.

In one aspect, the invention relates to a method of combination treatment. According to this method a LSD1 inhibitor and a second agent, which is an anticoagulant agent are administered to an individual (e.g. a human) in need of treatment wherein the individual has inflammation or an inflammatory disease or condition. In a more specific aspect, the anticoagulant agent is chosen from Heparin, warfarin, low molecular weight Heparins, acenocoumarol, phenprocoumon or other vitamin K antagonists, or direct thrombin inhibitor.

In one aspect, the invention relates to a method of combination treatment. According to this method a LSD1 inhibitor and a second agent, which is an anti-inflammatory agent are administered to an individual (e.g. a human) in need of treatment wherein the individual has inflammation or an inflammatory disease or condition. In specific aspect, the anti-inflammatory agent is chosen from a steroid, a NSAID, or a COX-2 selective inhibitor. In a more specific aspect, the anti-inflammatory agent is chosen from a steroid, a salicylate (e.g. aspirin, diflunisal, or salsalate), a propionic acid derivative (e.g., ibuprofen, naproxen, fenoprofen, ketoprofen, flurbiprofen, oxaprozin or loxoprofen), an acetic acid derivative (e.g., indomethacin, sulindac, etodolac, ketorolac, diclofenac or nabumetone), an enolic acid derivative (e.g., piroxicam, meloxicam, tenoxicam, droxicam, lornoxicam, or isoxicam), a fenamic acid derivative (e.g., mefenamic acid, meclofenamic acid, flufenamic acid or tolfenamic acid), a selective COX-2 inhibitor (e.g., celecoxib, rofecoxib, valdecoxib, parecoxib, lumiracoxib, etoricoxib, or firocoxib), a sulphonanilide (e.g. nimesulide) or licofelone.

In one aspect, the invention relates to a composition for combination treatment of inflammation or an inflammatory disease or condition. Accordingly, the pharmaceutical composition of this aspect comprises a LSD1 inhibitor and a second agent, which is an antiinflammatory agent, antiplatelet agent, or an anticoagulant agent, along with a pharmaceutically acceptable carrier or excipient. In one aspect, the second agent is an antiinflammatory agent, preferably an antiinflammatory agent chosen from a steroid, a NSAID, or a COX-2 selective inhibitor, more preferably an anti-inflammatory agent chosen from a steroid, a salicylate (e.g. aspirin, diflunisal, or salsalate), a propionic acid derivative (e.g., ibuprofen, naproxen, fenoprofen, ketoprofen, flurbiprofen, oxaprozin or loxoprofen), an acetic acid derivative (e.g., indomethacin, sulindac, etodolac, ketorolac, diclofenac or nabumetone), an enolic acid derivative (e.g., piroxicam, meloxicam, tenoxicam, droxicam, lornoxicam, or isoxicam), a fenamic acid derivative (e.g., mefenamic acid, meclofenamic acid, flufenamic acid or tolfenamic acid), a selective COX-2 inhibitor (e.g., celecoxib, rofecoxib, valdecoxib, parecoxib, lumiracoxib, etoricoxib, or firocoxib), a sulphonanilide (e.g. nimesulide) or licofelone In one aspect, the second agent is an antiplatelet agent, preferably an antiplatelet agent chosen from Aspirin, Clopidogrel, Prasugrel, Ticlopidine, Cilostazol, Abciximab, Eptifibatide, Tirofiban, Dipyridamole, Anagrelide, Hydroxyurea, or Epoprostenol. In one aspect, the second agent is an anticoagulant agent, preferably an anticoagulant agent chosen from Heparin, warfarin, low molecular weight Heparins, acenocoumarol, phenprocoumon, or a direct thrombin inhibitor.

In one aspect, the sufficient period of time for administering the LSD1 inhibitor is from five or more days to the individual, more preferably from five days to four years, even more preferably from five days to two years, yet even more preferably for fifteen days to two years, and again yet even more preferably from fifteen days to one year. In one aspect, the LSD1 inhibitor is administered daily in amount sufficient to yield a Cmax above the IC50 value for the LSD1 inhibitor. A person skilled in the art will appreciate that the Cmax should be above the IC50 value in the same species (e.g., in a human) in which the Cmax is to be measured.

The invention also relates to an LSD1 inhibitor for use in any of the above-described methods.

Accordingly, the invention relates to a LSD1 inhibitor for use in the treatment or prevention of inflammation or an inflammatory disease or condition. The invention also relates to a pharmaceutical composition comprising a LSD1 inhibitor and a pharmaceutically acceptable carrier for use in the treatment or prevention of inflammation or an inflammatory disease or condition. The inflammatory diseases or conditions to be treated or prevented in accordance with the invention are preferably selected from atherosclerosis, a respiratory inflammatory disorder (e.g. respiratory distress syndrome, asthma, COPD, bronchial hyperresponsiveness, bronchoconstriction, airway inflammation, airway remodeling or cystic fibrosis), chronic inflammatory bowel disease, ulcerative colitis, Crohn's disease, a chronic skin inflammatory disease (e.g. psoriasis or atopic dermatitis), mesangial glomerulonephritis, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, hepatic cirrhosis, or nephritis. More preferably, said inflammation or inflammatory disease or condition is atherosclerosis, respiratory distress syndrome, mesangial glomerulonephritis, chronic inflammatory bowel disease, ulcerative colitis, Crohn's disease, disseminated intravascular inflammation, allergic vasculitis, arthritis, rheumatoid arthritis, asthma, COPD, bronchial hyperresponsiveness, bronchoconstriction, airway inflammation, airway remodeling, cystic fibrosis, atopic dermatitis or psoriasis. In one particular embodiment, said inflammation or inflammatory disease or condition is atherosclerosis. In one particular embodiment, said inflammation or inflammatory disease or condition is a respiratory inflammatory disorder, such as respiratory distress syndrome, asthma, COPD, bronchial hyperresponsiveness, bronchoconstriction, airway inflammation, airway remodeling or cystic fibrosis. In one particular embodiment, said inflammation or inflammatory disease or condition is chronic inflammatory bowel disease, such as ulcerative colitis or Crohn's disease. In one embodiment, the invention relates to an LSD1 inhibitor (or a pharmaceutical composition comprising an LSD1 inhibitor and a pharmaceutically acceptable carrier) for use in the treatment or prevention of atherosclerosis, a respiratory inflammatory disorder (e.g. respiratory distress syndrome, asthma, COPD, bronchial hyperresponsiveness, bronchoconstriction, airway inflammation, airway remodeling or cystic fibrosis), chronic inflammatory bowel disease, ulcerative colitis, Crohn's disease, a chronic skin inflammatory disease (e.g. psoriasis or atopic dermatitis), mesangial glomerulonephritis, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, hepatic cirrhosis, or nephritis. In one embodiment, the invention relates to an LSD1 inhibitor (or a pharmaceutical composition comprising an LSD1 inhibitor and a pharmaceutically acceptable carrier) for use in the treatment or prevention of atherosclerosis, respiratory distress syndrome, mesangial glomerulonephritis, chronic inflammatory bowel disease, ulcerative colitis, Crohn's disease, disseminated intravascular inflammation, allergic vasculitis, arthritis, rheumatoid arthritis, asthma, COPD, bronchial hyperresponsiveness, bronchoconstriction, airway inflammation, airway remodeling, cystic fibrosis, atopic dermatitis or psoriasis. In one embodiment, the invention relates to an LSD1 inhibitor (or a pharmaceutical composition comprising an LSD1 inhibitor and a pharmaceutically acceptable carrier) for use in the treatment or prevention of a respiratory inflammatory disorder, such as respiratory distress syndrome, asthma, COPD, bronchial hyperresponsiveness, bronchoconstriction, airway inflammation, airway remodeling or cystic fibrosis. In one embodiment, the invention relates to an LSD1 inhibitor (or a pharmaceutical composition comprising an LSD1 inhibitor and a pharmaceutically acceptable carrier) for use in the treatment or prevention of atherosclerosis. In one embodiment, the invention relates to an LSD1 inhibitor (or a pharmaceutical composition comprising an LSD1 inhibitor and a pharmaceutically acceptable carrier) for use in the treatment or prevention of chronic inflammatory bowel disease, such as ulcerative colitis or Crohn's disease.

In another embodiment, the invention relates to an LSD1 inhibitor (or a pharmaceutical composition comprising an LSD1 inhibitor and a pharmaceutically acceptable carrier) for use in treating or preventing inflammation or an inflammatory disease or condition in an individual (e.g. in a human), wherein the LSD1 inhibitor is administered at an amount sufficient to reduce platelet levels in said individual.

In another embodiment the invention relates to an LSD1 inhibitor (or a pharmaceutical composition comprising an LSD1 inhibitor and a pharmaceutically acceptable carrier) for use in the treatment or prevention of a symptom of inflammation or an inflammatory disease or condition. In one aspect of this embodiment, said symptom is excessive or elevated platelet levels.

The present invention furthermore provides a LSD1 inhibitor to be administered in combination with one or more further therapeutic agents, in particular an antiinflammatory agent, an antiplatelet agent or an anticoagulant agent, for use in the treatment or prevention of inflammation or an inflammatory disease or condition, in particular for use for example in the treatment or prevention of atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis. The administration of the LSD1 inhibitor and the one or more further therapeutic agents may, e.g., be simultaneous/concomitant or sequential/separate. In one embodiment, the one or more further therapeutic agent is an anti-inflammatory agent, preferably chosen from a steroid, a NSAID, or a COX-2 selective inhibitor, more preferably chosen from a steroid, a salicylate (e.g. aspirin, diflunisal, or salsalate), a propionic acid derivative (e.g., ibuprofen, naproxen, fenoprofen, ketoprofen, flurbiprofen, oxaprozin or loxoprofen), an acetic acid derivative (e.g., indomethacin, sulindac, etodolac, ketorolac, diclofenac or nabumetone), an enolic acid derivative (e.g., piroxicam, meloxicam, tenoxicam, droxicam, lornoxicam, or isoxicam), a fenamic acid derivative (e.g., mefenamic acid, meclofenamic acid, flufenamic acid or tolfenamic acid), a selective COX-2 inhibitor (e.g., celecoxib, rofecoxib, valdecoxib, parecoxib, lumiracoxib, etoricoxib, or firocoxib), a sulphonanilide (e.g. nimesulide) or licofelone. In another embodiment, the one or more further therapeutic agent is an antiplatelet agent, preferably chosen from Aspirin, Clopidogrel, Prasugrel, Ticlopidine, Cilostazol, Abciximab, Eptifibatide, Tirofiban, Dipyridamole, Anagrelide, Hydroxyurea, or Epoprostenol. In another embodiment, the one or more further therapeutic agent is an anticoagulant agent, preferably chosen from Heparin, low molecular weight Heparins, a vitamin K antagonist such as warfarin, acenocoumarol or phenprocoumon, or a direct thrombin inhibitor.

The LSD1 inhibitor to be used in accordance with the present invention, in particular in the treatment or prevention of inflammation or an inflammatory disease or condition, is preferably a small molecule inhibitor of LSD1. In a preferred embodiment, the LSD1 inhibitor is a selective LSD1 inhibitor or a dual LSD1/MAO-B inhibitor. The LSD1 inhibitor to be used in accordance with the invention is preferably a 2-cyclylcyclopropan-1-amine compound, a phenelzine compound or a propargylamine compound, and is more preferably a 2-cyclylcyclopropan-1-amine compound. Said 2-cyclylcyclopropan-1-amine compound is preferably a 2-arylcyclopropan-1-amine compound Or a 2-heteroarylcyclopropan-1-amine compound, more preferably a 2-phenylcyclopropan-1-amine compound, a 2-pyridinylcyclopropan-1-amine compound or a 2-thiazolylcyclopropan-1-amine compound.

Thus the invention particularly relates to the following preferred embodiments:

1. A method of treating or preventing inflammation or an inflammatory disease or condition comprising administering to an individual a therapeutically effective amount of a LSD1 inhibitor.

2. The method as in 1, wherein the therapeutically effective amount of a LSD1 inhibitor is an amount sufficient to reduce platelets.

3. The method as in 1, wherein said inflammation or inflammatory disease or condition is chosen from atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis.

4. The method as in 1, wherein the LSD1 inhibitor is a selective LSD1 inhibitor.

5. The method as in 1, wherein the LSD1 inhibitor is a dual inhibitor of LSD1 and MAO-B.

6. The method as in 1, wherein the LSD1 inhibitor is an irreversible or a reversible amine oxidase inhibitor.

7. The method as in 1, wherein the LSD1 inhibitor is a phenylcyclopropylamine derivative or analog, a phenelzine derivative or analog, or a propargylamine derivative or analog.

8. The method as in 1, wherein the LSD1 inhibitor is a phenylcyclopropylamine derivative or analog.

9. The method as in 1, wherein the LSD1 inhibitor is a phenelzine derivative or analog.

10. The method as in 1, wherein the LSD1 inhibitor is a propargylamine derivative or analog.

11. The method as in 1, wherein said inflammation or inflammatory disease or condition, is caused by or associated with increased platelet counts.

12. The method as in 1, further comprising determining if the individual has inflammation or an inflammatory disease or condition.

13. The method as in 1, further comprising administering a second agent which is an anti-platelet agent, an anticoagulant agent or an anti-inflammatory agent to the individual.

14. The method as in 13, wherein said anti-inflammatory agent is chosen from steroids, Salicylates (Aspirin, Diflunisal, Salsalate), Propionic acid derivatives (e.g., Ibuprofen, Naproxen, Fenoprofen, Ketoprofen, Flurbiprofen, Oxaprozin or Loxoprofen), Acetic acid derivatives (e.g., Indomethacin, Sulindac, Etodolac, Ketorolac, Diclofenac or Nabumetone), Enolic acid derivatives (e.g., Piroxicam, Meloxicam, Tenoxicam, Droxicam, Lornoxicam, Isoxicam), Fenamic acid derivatives (e.g., Mefenamic acid, Meclofenamic acid, Flufenamic acid or Tolfenamic acid), Selective COX-2 inhibitors (e.g., Celecoxib, Rofecoxib, Valdecoxib, Parecoxib, Lumiracoxib, Etoricoxib, Firocoxib), Sulphonanilides (Nimesulide) and Licofelone.

15. A Pharmaceutical composition comprising a LSD1 inhibitor and a pharmaceutically acceptable carrier for use in any one of 1-14.

16. The LSD1 inhibitor of 15, wherein the LSD1 inhibitor is a selective LSD1 inhibitor.

17. The LSD1 inhibitor of 15, wherein the LSD1 inhibitor is a dual inhibitor of LSD1 and MAOB.

18. The LSD1 inhibitor of 15, wherein the LSD1 inhibitor is an irreversible or a reversible amine oxidase inhibitor.

19. The LSD1 inhibitor of 15, wherein the LSD1 inhibitor is a phenylcyclopropylamine derivative or analog, a phenelzine derivative or analog, or a propargylamine derivative or analog.

20. The LSD1 inhibitor of 15, wherein the LSD1 inhibitor is a phenylcyclopropylamine derivative or analog.

21. The LSD1 inhibitor of 15, wherein the LSD1 inhibitor is a phenelzine derivative or analog.

22. The LSD1 inhibitor of 15, wherein the LSD1 inhibitor is a propargylamine derivative or analog.

### BRIEF DESCRIPTION OF THE DRAWINGS

FIG. 1 Optimization of Selective LSD1 Inhibitors. FIG. 1 summarizes structure-activity relationship evolution of increased potency towards LSD1 as compared to MAO-A and/or MAO-B from compounds that were not selective (e.g., tranylcypromine, TCPA) to compounds that are selective inhibitors of LSD1 with IC50 values in the low nanomolar range.

FIG. 2 Optimization of Dual LSD1/MAO-B Inhibitors. FIG. 2 summarizes structure-activity relationship evolution of increased potency towards LSD1 and MAO-B as compared to MAO-A from compounds that were not selective for LSD1 and MAO-B (e.g., tranylcypromine, TCPA). The dual LSD1/MAO-B compounds have IC50 values for these two targets in the nanomolar range.

FIG. 3 Compound Dual-1 Increases Histone Methylation. FIG. 3 shows the results of a western blot stained for H3K4 methylation with SH-SY5Y cells grown in the presence of Compound Dual-1 (at 100 μM) or parnate (“PNT”) (at 250 μM) for one, two, and three days, showing that this compound, Dual-1, increases H3K4 methylation in cells in a time dependent manner.

### DETAILED DESCRIPTION OF THE INVENTION

The inventors have unexpectedly found that inhibitors of LSD1 reduce platelets (or other blood cells) in mammals and are therefore useful to treat or prevent inflammation or an inflammatory disease or condition, including in particular the inflammatory diseases/conditions described herein. It was found by the inventors that LSD1 inhibitors, selective LSD1 inhibitors, and dual inhibitors of LSD1 and MAO-B can be given to mammals at doses that are tolerated, and cause a reduction in platelets e.g., platelet count, as demonstrated in Example 5. Thus, the inventors have shown that LSD1 inhibitors inhibit platelet proliferation via an LSD1 mediated mechanism. This finding is significant since reduction of platelets or platelet count is medically very important and current treatments have undesirable side-effects and/or are marginally efficacious. Thus, the methods and compositions of the present invention can be useful for treating inflammation or inflammatory diseases or conditions, where the individual is resistant to or not effectively treated by current medications or that cannot comply with the treatment regimes employed with current medications. Additionally, the methods and compositions of the invention are useful for treating or preventing inflammation or an inflammatory disease or condition in combination with another therapeutic agent or drug, which is an anti-platelet agent or an anti-inflammatory agent or drug used in this clinical setting. Other advantages and more details of the invention are described below.

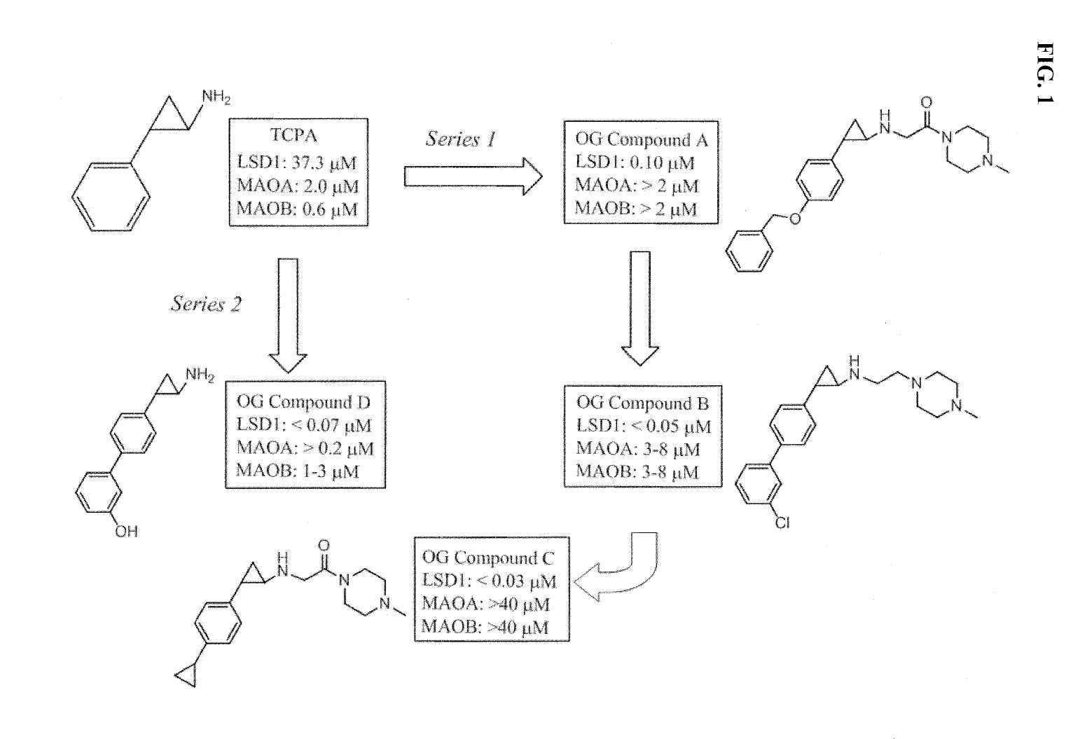

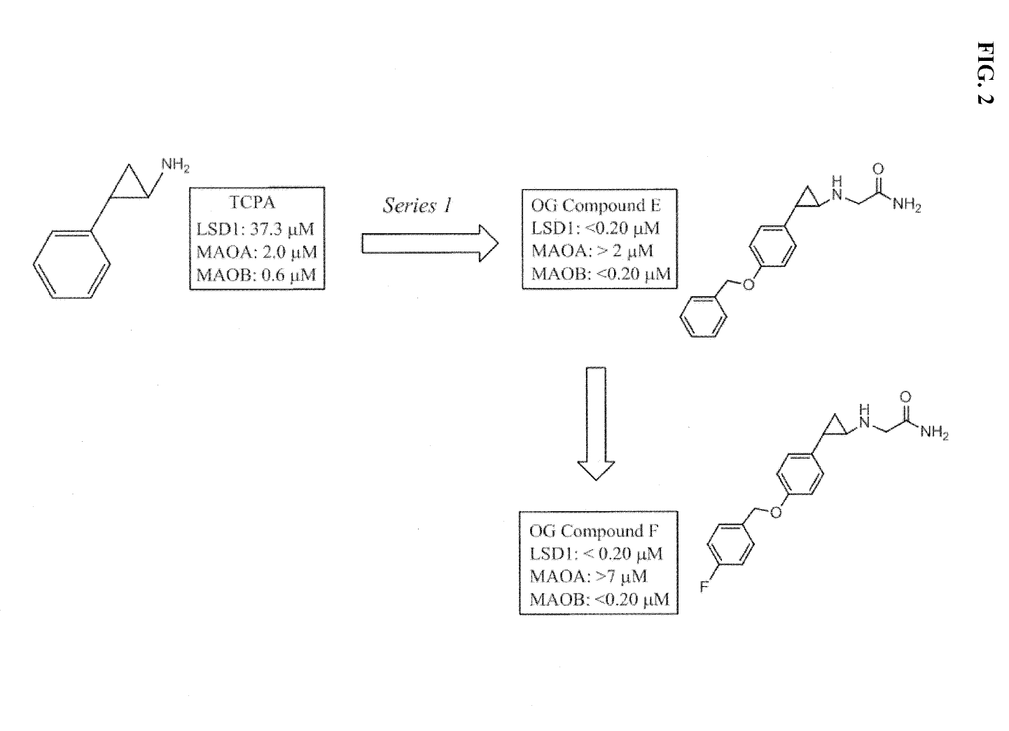

A medicinal chemistry effort undertaken by the applicant resulted in the synthesis and identification of small molecules, potent selective LSD1 inhibitors and potent dual inhibitors of LSD1 and MAO-B. This effort resulted in the identification of a number of compounds having different selectivities for LSD1, MAO-A, and MAO-B. See FIGS. 1 and 2.

Subsequent studies of some of the optimized compounds in a neural derived cell line and other cell lines indicated that both selective LSD1 inhibitors and dual inhibitors of LSD1 and MAOB can increase histone methylation levels at the cellular level indicating that these compounds inhibit cellular lysine demethylase activity.

Lastly the LSD1 inhibitors were to be able to be administered to mammals chronically at doses that are thought to achieve levels of the inhibitor sufficient for causing a biological effect.

As a result of these studies, a number of LSD1 inhibitors were shown to have activity in reducing platelets and other blood cells in vivo (see examples). Without being bound by theory, it is believed that LSD1 inhibitors, including selective LSD1 inhibitors and dual LSD1/MAOB inhibitors, such as 2-cyclylcyclopropan-1-amine compounds, phenelzine compounds, propargylamine compounds and other LSD1 inhibitors, inhibit platelet and blood cell proliferation and have use for treating inflammation or inflammatory diseases or conditions. More specifically, it is believed that LSD1 inhibitors, as a result of this invention, have use in treating or preventing atherosclerosis, a respiratory inflammatory disorder (e.g. respiratory distress syndrome, asthma, COPD, bronchial hyperresponsiveness, bronchoconstriction, airway inflammation, airway remodeling or cystic fibrosis), chronic inflammatory bowel disease, ulcerative colitis, Crohn's disease, a chronic skin inflammatory disease (e.g. psoriasis or atopic dermatitis), mesangial glomerulonephritis, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, hepatic cirrhosis, or nephritis, and in particular in treating or preventing atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis, psoriasis, or an associated disease or disorder.

### Methods of Treatment or Prevention and Disease:

The invention relates to methods of treatment or prevention of inflammation or inflammatory diseases or conditions with LSD1 inhibitors, and pharmaceutical compositions for treating or preventing inflammation or inflammatory diseases or conditions. In particular, the invention provides compositions and methods that can be used to reduce platelets or other blood cells and medical benefits derived therefrom.

Inflammation can be classified as either acute or chronic. Acute inflammation is the initial response of the body to harmful stimuli and is achieved by the increased movement of plasma and leukocytes from the blood into the injured tissues. Prolonged inflammation, known as chronic inflammation, can also lead to a host of diseases, such as hay fever, atherosclerosis, and rheumatoid arthritis.

In accordance with the present invention, LSD1 inhibitors can be used to treat inflammation of any tissue and organs of the body, including musculoskeletal inflammation, vascular inflammation, neural inflammation, digestive system inflammation, ocular inflammation, inflammation of the reproductive system, and other inflammation.

Musculoskeletal inflammation refers to any inflammatory condition of the musculoskeletal system, particularly those conditions affecting skeletal joints, including joints of the hand, wrist, elbow, shoulder, jaw, spine, neck, hip, knew, ankle, and foot, and conditions affecting tissues connecting muscles to bones such as tendons. Examples of musculoskeletal inflammation are arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular inflammation refers to inflammation of any structure of the eye, including the eye lids. Examples of ocular inflammation are blepharitis, blepharochalasis, conjunctivitis, dacryoadenitis, keratitis, keratoconjunctivitis sicca (dry eye), scleritis, trichiasis, and uveitis.

Examples of inflammation of the nervous system include encephalitis, Guillain-Barre syndrome, meningitis, neuromyotonia, narcolepsy, multiple sclerosis, myelitis and schizophrenia.

Examples of inflammation of the vasculature or lymphatic system include arthrosclerosis, arthritis, phlebitis, vasculitis, and lymphangitis.

Examples of inflammatory conditions of the digestive system are cholangitis, cholecystitis, enteritis, enterocolitis, gastritis, gastroenteritis, inflammatory bowel disease (such as Crohn's disease and ulcerative colitis), ileitis, and proctitis.

Examples of inflammatory conditions of the reproductive system include cervicitis, chorioamnionitis, endometritis, epididymitis, omphalitis, oophoritis, orchitis, salpingitis, tubo-ovarian abscess, urethritis, vaginitis, vulvitis, and vulvodynia.

Other inflammatory conditions which may be treated with an LSD1 inhibitor in accordance with the present invention include for example: atherosclerosis; respiratory inflammatory disorders such as asthma, chronic obstructive pulmonary disease (COPD), cystic fibrosis, respiratory distress syndrome, bronchial hyperresponsiveness, bronchoconstriction, airway inflammation or airway remodeling; mesangial glomerulonephritis, disseminated intravascular inflammation, allergic vasculitis, coronary artery disease, carotid artery disease, transplant vasculopathy, hepatic cirrhosis, nephritis or chronic skin inflammatory diseases such as atopic dermatits or psoriasis.

The present invention provides for the treatment or prevention of inflammation or an inflammatory disease or condition, comprising administering a LSD1 inhibitor to an individual. In particular, the invention provides compositions and methods that can be used to reduce platelets or other blood cells and medical benefits derived therefrom.

In one embodiment, the invention is the use of a LSD1 inhibitor for treating or preventing inflammation or an inflammatory disease or condition. In one aspect of this embodiment said inflammation or inflammatory disease or condition is associated with or caused by increased platelet count. In a related aspect, the invention is a method of treating or preventing inflammation or an inflammatory disease or condition, comprising administering a LSD1 inhibitor to an individual in need of such treatment or prevention. In yet another related aspect, the invention is a method of treating or preventing inflammation or an inflammatory disease or condition, comprising identifying an individual in need of such treatment or prevention and administering a LSD1 inhibitor to the individual. In a related aspect, the invention is a method of treating or preventing atherosclerosis, a respiratory inflammatory disorder (e.g. respiratory distress syndrome, asthma, COPD, bronchial hyperresponsiveness, bronchoconstriction, airway inflammation, airway remodeling or cystic fibrosis), chronic inflammatory bowel disease, ulcerative colitis, Crohn's disease, a chronic skin inflammatory disease (e.g. psoriasis or atopic dermatitis), mesangial glomerulonephritis, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, hepatic cirrhosis or nephritis, comprising administering a LSD1 inhibitor to an individual in need of such treatment. In a related aspect, the invention is a method of treating or preventing atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis, comprising administering a LSD1 inhibitor to an individual in need of such treatment. In a related aspect, the invention is a method of treating or preventing atherosclerosis, respiratory distress syndrome, mesangial glomerulonephritis, chronic inflammatory bowel disease, ulcerative colitis, Crohn's disease, disseminated intravascular inflammation, allergic vasculitis, arthritis, rheumatoid arthritis, asthma, COPD, bronchial hyperresponsiveness, bronchoconstriction, airway inflammation, airway remodeling, cystic fibrosis, atopic dermatitis or psoriasis, comprising administering a LSD1 inhibitor to an individual in need of such treatment. In a related aspect, the invention is a method of treating or preventing atherosclerosis, comprising administering a LSD1 inhibitor to an individual in need of such treatment. In a related aspect, the invention is a method of treating or preventing a respiratory inflammatory disorder, such as respiratory distress syndrome, asthma, COPD, bronchial hyperresponsiveness, bronchoconstriction, airway inflammation, airway remodeling or cystic fibrosis, comprising administering a LSD1 inhibitor to an individual in need of such treatment. In a related aspect, the invention is a method of treating or preventing chronic inflammatory bowel disease, such as ulcerative colitis or Crohn's disease, comprising administering a LSD1 inhibitor to an individual in need of such treatment. In another aspect, the invention is the use of a LSD1 inhibitor in an amount sufficient to modulate LSD1 activity for treating or preventing inflammation or an inflammatory disease or condition, in an individual. In one aspect of the method described in this paragraph, the method further comprises determining if the individual has inflammation or an inflammatory disease or condition, associated with or caused by increased platelets counts. In one aspect, the LSD1 inhibitor described in this paragraph is a small molecule inhibitor of LSD1. In one aspect, the LSD1 inhibitor described in this paragraph is a selective inhibitor of LSD1. In one aspect, the LSD1 inhibitor described in this paragraph is a selective inhibitor of LSD1 and MAO-B (e.g. a dual inhibitor of LSD1 and MAO-B). In one aspect, the LSD1 inhibitor described in this paragraph is an irreversible or a reversible amine oxidase inhibitor. In one aspect, the amine oxidase inhibitor of this paragraph is a phenylcyclopropylamine derivative or analog, a phenelzine derivative or analog, or a propargylamine derivative or analog. In one aspect, the LSD1 inhibitor described in this paragraph is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound.

In one embodiment, the invention is the use of a LSD1 inhibitor for treating or preventing inflammation or an inflammatory disease or condition. In a related aspect, the invention is a method of treating or preventing inflammation or inflammatory diseases or conditions, comprising administering a LSD1 inhibitor to an individual. In another related aspect, the invention is a method of treating or preventing inflammation or an inflammatory disease or condition, comprising administering a LSD1 inhibitor to an individual in need of such treatment or prevention. In yet another related aspect, the invention is a method of treating or preventing inflammation or an inflammatory disease or condition, comprising identifying an individual in need of such treatment or prevention and administering a LSD1 inhibitor to the individual. In a related aspect, the invention is the use of a LSD1 inhibitor in an amount sufficient to modulate LSD1 activity for treating or preventing atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis, in an individual having any one of these diseases or conditions. In one aspect of the method described in this paragraph, the method further comprises determining if the individual has an inflammatory disease or condition. In one aspect, the LSD1 inhibitor described in this paragraph is a small molecule inhibitor of LSD1. In one aspect, the LSD1 inhibitor described in this paragraph is a selective inhibitor of LSD1. In one aspect, the LSD1 inhibitor described in this paragraph is a selective inhibitor of LSD1 and MAO-B (e.g., a dual inhibitor of LSD1 and MAO-B). In one aspect, the LSD1 inhibitor described in this paragraph is an irreversible or a reversible amine oxidase inhibitor. In one aspect, the amine oxidase inhibitor of this paragraph is a phenylcyclopropylamine derivative or analog, a phenelzine derivative or analog, or a propargylamine derivative or analog. In one aspect, the LSD1 inhibitor described in this paragraph is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound.

In one embodiment, the invention is the use of an amount of an LSD1 inhibitor sufficient for reducing platelets, for the treatment or prevention of inflammation or an inflammatory disease or condition. In a related aspect, the invention provides a method of treating or preventing inflammation or an inflammatory disease or condition, in an individual in need of such treatment by administering a therapeutically effective amount of a LSD1 inhibitor, wherein the therapeutically effect amount is an amount sufficient to reduce platelets. In yet another related aspect, the invention is a method of treating or preventing inflammation or an inflammatory disease or condition, comprising identifying an individual in need of such treatment or prevention and administering a LSD1 inhibitor, in an amount sufficient to reduce platelets, to the individual. In a related aspect, the invention is the use of a LSD1 inhibitor, in an amount sufficient to reduce platelets, for treating or preventing inflammation or an inflammatory disease or condition. In a related aspect, the invention is the use of a LSD1 inhibitor, in an amount sufficient to reduce platelets, for treating or preventing atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis and psoriasis, or a related disease, in an individual having any of these diseases or conditions. In one aspect of the method described in this paragraph, the method further comprises determining if the individual has inflammation or an inflammatory disease or condition. In one aspect, the LSD1 inhibitor described in this paragraph is a small molecule inhibitor of LSD1. In one aspect, the LSD1 inhibitor described in this paragraph is a selective inhibitor of LSD1. In one aspect, the LSD1 inhibitor described in this paragraph is a selective inhibitor of LSD1 and MAO-B. In one aspect, the LSD1 inhibitor described in this paragraph is an irreversible or a reversible amine oxidase inhibitor. In one aspect, the amine oxidase inhibitor of this paragraph is a phenylcyclopropylamine derivative or analog, a phenelzine derivative or analog, or a propargylamine derivative or analog. In one aspect, the LSD1 inhibitor described in this paragraph is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound.

In one embodiment, the invention is the use of a LSD1 inhibitor for treating or preventing inflammation or an inflammatory disease or condition. In a related aspect, the invention is a method of treating or preventing inflammation or an inflammatory disease or condition, comprising administering a LSD1 inhibitor to an individual. In another related aspect, the invention is a method of treating or preventing inflammation or an inflammatory disease or condition, comprising administering a therapeutically effective amount of a LSD1 inhibitor to an individual in need of such treatment. In yet another related aspect, the invention is a method of treating or preventing inflammation or an inflammatory disease or condition, comprising identifying an individual in need of such treatment or prevention and administering a LSD1 inhibitor to the individual. In one aspect, the invention is the use of a LSD1 inhibitor in an amount sufficient to modulate LSD1 activity for treating or preventing inflammation or an inflammatory disease or condition. In a related aspect, the invention is the use of a LSD1 inhibitor in an amount sufficient to modulate LSD1 activity for treating or preventing atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis, in an individual having any of these diseases or conditions. In one aspect of the method described in this paragraph, the method further comprises determining if the individual has inflammation or an inflammatory disease or condition. In one aspect, the LSD1 inhibitor described in this paragraph is a small molecule inhibitor of LSD1. In one aspect, the LSD1 inhibitor described in this paragraph is a selective inhibitor of LSD1. In one aspect, the LSD1 inhibitor described in this paragraph is a selective inhibitor of LSD1 and MAO-B. In one aspect, the LSD1 inhibitor described in this paragraph is an irreversible or a reversible amine oxidase inhibitor. In one aspect, the amine oxidase inhibitor of this paragraph is a phenylcyclopropylamine derivative or analog, a phenelzine derivative or analog, or a propargylamine derivative or analog. In one aspect, the LSD1 inhibitor described in this paragraph is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound.

The patient, subject, or individual, such as the individual in need of treatment or prevention, may be, e.g., a eukaryote, an animal, a vertebrate animal, a mammal, a rodent (e.g., a guinea pig, a hamster, a rat, a mouse), a murine (e.g., a mouse), a canine (e.g., a dog), a feline (e.g., a cat), an equine (e.g., a horse), a primate, a simian (e.g., a monkey or ape), a monkey (e.g., a marmoset, a baboon), an ape (e.g., gorilla, chimpanzee, orangutan, gibbon), or a human. The meaning of the terms “eukaryote,” “animal,” “mammal,” etc., is well known in the art and can, for example, be deduced from Wehner and Gehring (1995; Thieme Verlag). In the context of this invention, it is particularly envisaged that animals are to be treated which are economically, agronomically or scientifically important. Scientifically important organisms include, but are not limited to, mice, rats, rabbits, fruit flies like Drosophila melagonaster and nematodes like Caenorhabditis elegans. Non-limiting examples of agronomically important animals are sheep, cattle and pig, while, for example, cats and dogs may be considered as economically important animals. Preferably, the individual/subject/patient is a mammal; more preferably, the individual/subject/patient is a human.

As used herein, the term “treating a disease or disorder” refers to a slowing of or a reversal of the progress of the disease. Treating a disease or disorder includes treating a symptom and/or reducing the symptoms of the disease.

As used herein, the term “preventing a disease or disorder” refers to a slowing of the disease or of the onset of the disease or the symptoms thereof. Preventing a disease or disorder can include stopping the onset of the disease or symptoms thereof.

As used herein, “LSD1 inhibitor” refers to a molecule that directly or indirectly lowers or downregulates a biological activity of Lysine Dependent Demethylase 1 (LSD1). A LSD1 inhibitor may be any member of a class of compounds (e.g. a small molecule, or an antibody or a fragment or derivative of such antibody such as a Fab fragment or a single chain antibody such as a scFv) that binds LSD1 and inhibits a biological activity (e.g. demethylase activity) of a LSD1 protein or a protein complex in which LSD1 exerts its function (e.g. LSD1 being complexed to co-REST and/or other protein members of the nucleosome). A LSD1 inhibitor may also be any member of a class of compounds that decreases the expression of a nucleic acid encoding a LSD1 protein (e.g. an inhibitory nucleic acid, RNAi, such as a small hairpin RNA). Preferably, a LSD1 inhibitor is a compound that exhibits LSD1-inhibitory activity in the LSD1 biological assay disclosed in Example 1. The skilled person is able to determine whether a compound would qualify as LSD1 inhibitor in such assay. Preferably, a LSD1 inhibitor is a compound that exhibits more than 50% inhibition of LSD1 activity in the LSD1 assay of example 1 at 50 μM, more preferably one that exhibits more than 50% inhibition of LSD1 activity in the LSD1 assay of example 1 at 10 μM, still more preferably one that exhibits more than 50% inhibition of LSD1 activity in the LSD1 assay of example 1 at 1 μM, and even more preferably one that exhibits more than 50% inhibition of LSD1 activity in the LSD1 assay of example 1 at a concentration of 0.5 μM or less.

As used herein “a small molecule inhibitor of LSD1” (or “small molecule” as used in relation to an LSD1 inhibitor) refers to an LSD1 inhibitor having a molecular weight of less than 1000 daltons, preferably less than 700 daltons.

As used herein, the term “selective LSD1 inhibitor”, “LSD1 selective inhibitor” or “selective inhibitor of LSD1” refers to an LSD1 inhibitor which preferably has an IC50 value for LSD1 that is at least two-fold lower than its IC50 values for MAO-A and MAO-B. More preferably, a selective LSD1 inhibitor has an IC50 value for LSD1 which is at least five-fold lower than its IC50 values for MAO-A and MAO-B. Even more preferably, a selective LSD1 inhibitor has an IC50 value for LSD1 which is at least ten-fold lower than its IC50 values for MAO-A and MAO-B. Even more preferably, a selective LSD1 inhibitor has an IC50 value for LSD1 which is at least 20-fold lower than its IC50 values for MAO-A and MAO-B. Even more preferably, a selective LSD1 inhibitor has an 1050 value for LSD1 which is at least 50-fold lower than its IC50 values for MAO-A and MAO-B. Even more preferably, a selective LSD1 inhibitor has an IC50 value for LSD1 which is at least 100-fold lower than its IC50 values for MAO-A and MAO-B. The ability of a compound to inhibit LSD1 and its IC50 values for LSD1, MAO-A and MAO-B are preferably to be determined in accordance with the experimental protocol described in Example 1.

As used herein, the terms “selective inhibitor of LSD1 and MAOB”, “dual LSD1/MAO-B inhibitor”, “LSD1/MAO-B inhibitor”, “dual LSD1/MAOB selective inhibitor”, “dual inhibitor selective for LSD1 and MAO-B” or “dual inhibitor of LSD1 and MAO-B” are used interchangeably and refer to an LSD1 inhibitor which preferably has IC50 values for LSD1 and MAO-B which are at least two-fold lower than its IC50 value for MAO-A. More preferably, a dual LSD1/MAO-B selective inhibitor has IC50 values for LSD1 and MAO-B which are at least five-fold lower than its IC50 value for MAO-A. Even more preferably, a dual LSD1/MAO-B selective inhibitor has IC50 values for LSD1 and MAO-B which are at least ten-fold lower than its 1050 value for MAO-A. Even more preferably, a dual LSD1/MAO-B selective inhibitor has IC50 values for LSD1 and MAO-B which are at least 20-fold lower than its IC50 value for MAO-A. The ability of a compound to inhibit LSD1 and MAO-B and its IC50 values for LSD1, MAO-A and MAO-B are preferably to be determined in accordance with the experimental protocol described in Example 1.

As used herein, a “platelet reducing effective amount of an LSD1 inhibitor” is an amount of said LSD1 inhibitor sufficient to reduce platelet levels.

Accordingly, “a platelet reducing effective amount” or “an amount sufficient to reduce platelets” also includes an amount of a substance or compound, e.g., an LSD1 inhibitor, which when administered to an individual over a certain time causes a decrease in platelet counts as compared to a standard value or range or refers to a lessening or decrease of platelet counts in an individual where the platelet count is elevated, e.g., due to inflammation or an inflammatory disease or condition.

Methods to measure platelet (or other blood cell) levels are well known in the art and they can be used to determine the ability of a compound, such as an LSD1 inhibitor, to reduce blood cell, particularly platelet levels. For example, the compound to be assayed for platelet reducing activity can be administered by the desired route of administration and then blood samples are collected in a tube containing an anticoagulant agent (such as EDTA, citrate and the like) and analyzed in a standard hematology analyzer. Said analyzer routinely uses flow cytometry and electric detectors and electric impedance for cell counting and identification. Manual counts can also be used for complete blood counts. The skilled person is able to determine based on the data obtained from such an assay whether a compound would qualify as a compound that reduces platelet or other blood cell levels. A suitable assay to measure the ability of a compound to reduce platelet levels is, for instance, that disclosed in Example 5. Preferably, a compound is regarded as exhibiting platelet reducing activity if platelet levels are reduced by 20% or more as compared to a control sample using the method disclosed in Example 5.

In the context of this invention, a “reduction in platelets” (or other blood cells) or a “reduction of platelet levels” may, accordingly, comprise the reduction in platelet/cell count. The term “reducing platelets” or “reducing platelet count” may thus refer to a decrease in platelet counts, particularly a decrease in platelet counts as compared to a standard value or range, or may also refer to a lessening or decrease of platelet counts in an individual where the platelet count is elevated, e.g., due to inflammation or an inflammatory disease or condition. As illustrated in the appended examples, the compounds of the present invention are surpassingly capable of reducing cell count/cell levels, in particular of blood cells and most particularly of platelets. Accordingly, the LSD1 inhibitors as provided herein are useful in reducing (blood) cell counts/levels, in particular in reducing counts/levels of platelets. A “reduction in count/level” in this respect can be measured by means and methods provided herein and in the appended examples. A “reduction in (blood) cell and/or platelet levels” and/or a “reduction of (blood) cell and/or platelet counts” can comprise the measurement of a given biological sample, like a blood sample, derived from a patient in need of medical intervention as provided herein in comparison to a given control sample or control samples or as compared to standard references or standard reference values. Such a control sample or such control samples may comprise corresponding samples from healthy individuals or from defined diseased individuals (for example individuals suffering from or being prone to suffer from inflammatory disorders). Such a control sample may also comprise a biological sample from the same individual to be assessed (like the patient) whereby said sample was taken at an earlier or a later stage when said individual was or is healthy or diseased (i.e. before, during or after medical intervention as disclosed herein). In the context of this invention the “platelet reduction” to be achieved with the compounds of the present invention is preferably a reduction of at least 10%, more preferably of at least 20%, and even more preferably of at least 30% as compared to a control sample or as compared to standard references or standard reference values.

As used herein, the term “increased platelet count” refers to a platelet count higher than the normal platelet count. The normal platelet count in adults ranges from 150 to 450 K/μL.

As used herein, the term “unit dosage form” refers to a physically discrete unit, such as a capsule or tablet suitable as a unitary dosage for a human patient. Each unit contains a predetermined quantity of a LSD1 inhibitor, which was discovered or believed to produce the desired pharmacokinetic profile which yields the desired therapeutic effect. The dosage unit is composed of a LSD1 inhibitor in association with at least one pharmaceutically acceptable carrier, salt, excipient, or combination thereof.

In another aspect, the invention is a method of treating inflammation or inflammatory diseases, or conditions, comprising identifying an individual in need of such treatment and administering to the individual for a sufficient period of time an amount of a LSD1 inhibitor, preferably a selective LSD1 inhibitor, sufficient to treat or prevent inflammation or an inflammatory disease or condition. In a related aspect, the invention is the use of a LSD1 inhibitor, preferably a selective LSD1 inhibitor, in an amount sufficient to modulate LSD1 activity for treating or preventing inflammation or an inflammatory disease or condition. In one embodiment of this aspect, the amount of LSD1 inhibitor, preferably a selective LSD1 inhibitor, administered is sufficient to modulate or inhibit LSD1 activity while not substantially inhibiting MAO-A activity, thereby avoiding or reducing side-effects associated with administration of MAO-A inhibitors. In a specific aspect of this embodiment, preferably the amount of LSD1 inhibitor, preferably a selective LSD1 inhibitor, administered per day to a human is from about 0.01 mg to about 500 mg per day. More preferably the amount of LSD1 inhibitor administered per day to a human is from about 0.01 mg to about 200 mg per day or is a pharmaceutical composition formulated in such a way as to deliver this amount of free base equivalent (or free acid equivalent depending on the parent molecule). Preferably, the LSD1 inhibitor is administered or formulated to be administered for five or more days to the individual, more preferably from five days to four years, even more preferably from five day to two years, yet even more preferably for fifteen days to two years, and again yet even more preferably from fifteen days to one year. It is noted that in this context administration for, e.g., five or more days, means an amount over a time sufficient to cause pharmacologic inhibition of LSD1 over this period of time and this does not necessarily mean administration of compound every day or only once per day. Depending on the PKPD and ADME properties of the inhibitors, a suitable amount and dosing regimen can be determined by a skilled practitioner in view of this disclosure.

In one aspect, the invention is a method of treating or preventing inflammation or an inflammatory disease or condition, comprising identifying an individual in need of such treatment and administering to the individual for a sufficient period of time an amount of a dual LSD1/MAO-B inhibitor sufficient to treat or prevent inflammation or an inflammatory disease or condition. In a related aspect, the invention is the use of a dual LSD1/MAO-B inhibitor in an amount sufficient to modulate LSD1 activity for treating or preventing inflammation or an inflammatory disease or condition. In a specific aspect, treating or preventing inflammation or inflammatory disease or condition, comprises reducing platelets. In one embodiment of this aspect, the amount of a dual LSD1/MAO-B inhibitor administered is sufficient to modulate or inhibit LSD1 and MAO-B activity while not substantially inhibiting MAO-A activity, thereby avoiding or reducing side-effects associated with administration of MAO-A inhibitors. In a specific aspect of this embodiment, preferably the amount of dual LSD1/MAOB inhibitor administered per day to a human is from about 0.01 mg to about 500 mg per day (e.g., 0.5 mg to about 500 mg per day). More preferably the amount of dual LSD1/MAO-B inhibitor administered per day to a human is from about 0.01 mg to about 200 mg per day (e.g., 0.5 mg to about 200 mg per day) or is a pharmaceutical composition formulated in such a way as to deliver this amount of free base equivalent (or free acid equivalent depending on the parent molecule). In one embodiment of this aspect, the amount of dual LSD1/MAO-B inhibitor administered is sufficient to modulate or inhibit LSD1/MAO-B activity while not substantially inhibiting MAO-A activity, thereby avoiding or reducing side-effects associated with administration of MAO-A inhibitors. Preferably, the dual LSD1/MAO-B inhibitor is administered or formulated to be administered for five or more days to the individual, more preferably from five days to four years, even more preferably from five days to two years, yet even more preferably for fifteen days to two years, and again yet even more preferably from fifteen days to one year. It is noted that in this context administration for, e.g., five or more days, means an amount over a time sufficient to cause pharmacologic inhibition of LSD1 and MAO-B over this period of time and this does not necessarily mean administration of compound every day or only once per day. Depending on the PK/PD and ADME properties of the inhibitors, a suitable amount and dosing regimen can be determined by a skilled practitioner in view of this disclosure.

In one embodiment, the invention is a method of treating or preventing inflammation or an inflammatory disease or condition, comprising identifying an individual in need of such treatment and administering to the individual a LSD1 inhibitor and a second agent, which is an anti-platelet drug or agent to treat or prevent inflammation or an inflammatory disease or condition. In a related aspect, the invention is the use of a LSD1 inhibitor and said anti-platelet drug in an amount sufficient for treating or preventing inflammation or an inflammatory disease or condition. In a specific aspect, treating or preventing inflammation or an inflammatory disease or condition comprises inhibiting platelets via LSD1 and inhibiting inflammation or an inflammatory disease or condition with a second agent, which is an anti-platelet drug chosen from Aspirin, Clopidogrel, Prasugrel, Ticlopidine, Cilostazol, Abciximab, Eptifibatide, Tirofiban, Dipyridamole, Anagrelide, Hydroxyurea, or Epoprostenol. Other suitable antiplatelet agents include Ticagrelor or thromboxane inhibitors. In one embodiment of this aspect, the amount of said anti-platelet drug is sufficient to prevent or treat inflammation or an inflammatory disease or condition. In one embodiment of this aspect, the amount of said anti-platelet drug administered is sufficient to prevent or treat inflammation or an inflammatory disease or condition while avoiding or reducing side-effects associated with administration of higher doses of said anti-platelet drug. In one aspect, the anti-platelet agent is Aspirin. In one aspect, the anti-platelet agent is Clopidogrel. In one aspect, the anti-platelet agent is ticlopidine In a specific aspect of this embodiment, preferably the amount of LSD1 inhibitor administered per day to a human is from about 0.01 mg to about 500 mg per day (e.g., from about 0.5 mg to about 500 mg per day). More preferably the amount of LSD1 inhibitor administered per day to a human is from about 0.01 mg to about 200 mg per day (e.g., from about 0.5 mg to about 200 mg per day) or is a pharmaceutical composition formulated in such a way as to deliver this amount of free base equivalent (or free acid equivalent depending on the parent molecule). In one embodiment of this aspect, the amount of the anti-platelet agent administered to the individual is from 0.050 to 1000 mg daily. More preferably, the amount of the anti-platelet drug is administered to the individual is from 0.050 to 500 mg daily. Even more preferably, the amount of the anti-platelet drug administered to the individual is from 0.050 to 200 mg daily. Depending on the PK/PD and ADME properties of the inhibitors, a suitable amount and dosing regimen can be determined by a skilled practitioner in view of this disclosure.

In one embodiment, the invention is a method of treating or preventing inflammation or an inflammatory disease or condition, comprising identifying an individual in need of such treatment and administering to the individual a LSD1 inhibitor and a second agent, which is an anticoagulant agent to treat or prevent inflammation or an inflammatory disease or condition. In a related aspect, the invention is the use of a LSD1 inhibitor and said anticoagulant agent in an amount sufficient for treating or preventing inflammation or an inflammatory disease or condition. In a specific aspect, treating or preventing inflammation or an inflammatory disease or condition, comprises inhibiting platelets via LSD1 and inhibiting inflammation or an inflammatory disease or condition, with a second agent which is an anticoagulant agent chosen from Heparin, low molecular weight Heparins, vitamin K antagonists such as Warfarin, acenocoumarol or phenprocoumon, or direct thrombin inhibitors. In one embodiment of this aspect, the amount of said anticoagulant agent is sufficient to prevent or treat inflammation or an inflammatory disease or condition. In one embodiment of this aspect, the amount of said anticoagulant drug administered is sufficient to prevent or treat inflammation or an inflammatory disease or condition, while avoiding or reducing side-effects associated with administration of higher doses of the anticoagulant agent. In one aspect, the anticoagulant agent is Heparin. In one aspect, the anticoagulant agent is a vitamin K antagonist. In one aspect, the anticoagulant agent is a warfarin. In a specific aspect of this embodiment, preferably the amount of LSD1 inhibitor administered per day to a human is from about 0.01 mg to about 500 mg per day (e.g., from about 0.5 mg to about 500 mg per day). More preferably the amount of LSD1 inhibitor administered per day to a human is from about 0.01 mg to about 200 mg per day (e.g., from about 0.5 mg to about 200 mg per day) or is a pharmaceutical composition formulated in such a way as to deliver this amount of free base equivalent (or free acid equivalent depending on the parent molecule). In one embodiment of this aspect, the amount of the anticoagulant drug administered to the individual is from 0.050 to 1000 mg daily. More preferably, the amount of the anticoagulant agent is administered to the individual is from 0.050 to 500 mg daily. Even more preferably, the amount of the anticoagulant drug administered to the individual is from 0.050 to 200 mg daily. Depending on the PK/PD and ADME properties of the inhibitors, a suitable amount and dosing regimen can be determined by a skilled practitioner in view of this disclosure.

In one embodiment, the invention is a method of treating or preventing inflammation or an inflammatory disease or condition, comprising identifying an individual in need of such treatment and administering to the individual a LSD1 inhibitor and a second agent, which is an anti-inflammatory agent to treat or prevent inflammation or an inflammatory disease or condition. In a related aspect, the invention is the use of a LSD1 inhibitor and said anti-inflammatory agent in an amount sufficient for treating or preventing inflammation or an inflammatory disease or condition. In a specific aspect, treating or preventing inflammation or an inflammatory disease or condition comprises inhibiting platelets via LSD1 and inhibiting inflammation or an inflammatory disease or condition, with a second agent, which is an anti-inflammatory agent chosen from a steroid, a NSAID, or a COX-2 selective inhibitor, more preferably chosen from a steroid, a salicylate (e.g., aspirin, diflunisal, or salsalate), a propionic acid derivative (e.g., ibuprofen, naproxen, fenoprofen, ketoprofen, flurbiprofen, oxaprozin or loxoprofen), an acetic acid derivatives (e.g., indomethacin, sulindac, etodolac, ketorolac, diclofenac or nabumetone), an enolic acid derivative (e.g., piroxicam, meloxicam, tenoxicam, droxicam, lornoxicam, or isoxicam), a fenamic acid derivative (e.g., mefenamic acid, meclofenamic acid, flufenamic acid or tolfenamic acid), a selective COX-2 inhibitor (e.g., celecoxib, rofecoxib, valdecoxib, parecoxib, lumiracoxib, etoricoxib, or firocoxib) a sulphonanilide (e.g. mimesulide) or licofelone. In one embodiment of this aspect, the amount of said anti-inflammatory agent is sufficient to prevent or treat inflammation or an inflammatory disease or condition. In one embodiment of this aspect, the amount of said anti-inflammatory agent administered is sufficient to prevent or treat inflammation or an inflammatory disease or condition, while avoiding or reducing side-effects associated with administration of higher doses of the anti-inflammatory agent. In one aspect, the anti-inflammatory agent is an NSAID. In one aspect, the anti-inflammatory agent is a steroid. In one aspect, the anti-inflammatory agent is a COX-2 inhibitor. In one aspect, the anti-inflammatory agent is a propionic acid derivative. In a specific aspect of this embodiment, preferably the amount of LSD1 inhibitor administered per day to a human is from about 0.5 mg to about 500 mg per day. More preferably the amount of LSD1 inhibitor administered per day to a human is from about 0.5 mg to about 200 mg per day or is a pharmaceutical composition formulated in such a way as to deliver this amount of free base equivalent (or free acid equivalent depending on the parent molecule). In one embodiment of this aspect, the amount of the anti-inflammatory agent administered to the individual is from 0.050 to 1000 mg daily. More preferably, the amount of the anti-inflammatory agent is administered to the individual is from 0.050 to 500 mg daily. Even more preferably, the amount of the anti-inflammatory agent administered to the individual is from 0.050 to 200 mg daily. Depending on the PK/PD and ADME properties of the inhibitors, a suitable amount and dosing regimen can be determined by a skilled practitioner in view of this disclosure.

The invention also relates to an LSD1 inhibitor for use in any of the above-described methods.

Accordingly, the invention relates to an LSD1 inhibitor (or a pharmaceutical composition comprising an LSD1 inhibitor and a pharmaceutically acceptable carrier) for use in treating or preventing inflammation or an inflammatory disease or condition. In one embodiment, the inflammation or inflammatory disease or condition is atherosclerosis, a respiratory inflammatory disorder (e.g. respiratory distress syndrome, asthma, COPD, bronchial hyperresponsiveness, bronchoconstriction, airway inflammation, airway remodeling or cystic fibrosis), chronic inflammatory bowel disease, ulcerative colitis, Crohn's disease, a chronic skin inflammatory disease (e.g. psoriasis or atopic dermatitis), mesangial glomerulonephritis, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, hepatic cirrhosis, or nephritis. In one embodiment, the inflammation or inflammatory disease or condition is atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis. In one embodiment, the inflammation or inflammatory disease or condition is atherosclerosis, respiratory distress syndrome, mesangial glomerulonephritis, chronic inflammatory bowel disease, ulcerative colitis, Crohn's disease, disseminated intravascular inflammation, allergic vasculitis, arthritis, rheumatoid arthritis, asthma, COPD, bronchial hyperresponsiveness, bronchoconstriction, airway inflammation, airway remodeling, cystic fibrosis, atopic dermatitis or psoriasis. In one particular embodiment, said inflammation or inflammatory disease or condition is atherosclerosis. In one particular embodiment, said inflammation or inflammatory disease or condition is a respiratory inflammatory disorder, such as respiratory distress syndrome, asthma, COPD, bronchial hyperresponsiveness, bronchoconstriction, airway inflammation, airway remodeling or cystic fibrosis. In one particular embodiment, said inflammation or inflammatory disease or condition is chronic inflammatory bowel disease, such as ulcerative colitis or Crohn's disease. In one aspect, the LSD1 inhibitor is a small molecule inhibitor of LSD1. In one aspect, the LSD1 inhibitor is a selective inhibitor of LSD1. In one aspect, the LSD1 inhibitor is a selective inhibitor of LSD1 and MAOB (i.e. a dual LSD1/MAO-B inhibitor). In one aspect, the LSD1 inhibitor is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound.

The invention also relates to an LSD1 inhibitor (or a pharmaceutical composition comprising an LSD1 inhibitor and a pharmaceutically acceptable carrier) for use in treating or preventing inflammation or an inflammatory disease or condition in an individual (e.g. in a human), wherein the LSD1 inhibitor is administered at an amount sufficient to reduce platelet levels in said individual. In one embodiment, the inflammation or inflammatory disease or condition is chosen from atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis. In one aspect, the LSD1 inhibitor is a small molecule inhibitor of LSD1. In one aspect, the LSD1 inhibitor is a selective inhibitor of LSD1. In one aspect, the LSD1 inhibitor is a selective inhibitor of LSD1 and MAOB (i.e. a dual LSD1/MAO-B inhibitor). In one aspect, the LSD1 inhibitor is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound, or a propargylamine derivative or analog.

In another embodiment the invention relates to an LSD1 inhibitor (or a pharmaceutical composition comprising an LSD1 inhibitor and a pharmaceutically acceptable carrier) for use in the treatment or prevention of a symptom of inflammation or an inflammatory disease or condition. In one aspect of this embodiment, said symptom is excessive or elevated platelet levels. In one aspect, the inflammation or inflammatory disease or condition is chosen from atherosclerosis, a respiratory inflammatory disorder (e.g. respiratory distress syndrome, asthma, COPD, bronchial hyperresponsiveness, bronchoconstriction, airway inflammation, airway remodeling or cystic fibrosis), chronic inflammatory bowel disease, ulcerative colitis, Crohn's disease, a chronic skin inflammatory disease (e.g. psoriasis or atopic dermatitis), mesangial glomerulonephritis, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, hepatic cirrhosis, or nephritis. In one aspect, the inflammation or inflammatory disease or condition is chosen from atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis. In one aspect, the LSD′ inhibitor is a small molecule inhibitor of LSD1. In one aspect, the LSD1 inhibitor is a selective inhibitor of LSD1. In one aspect, the LSD1 inhibitor is a selective inhibitor of LSD1 and MAOB (i.e. a dual LSD1/MAO-B inhibitor). In one aspect, the LSD1 inhibitor is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, 2-pyridinylcyclopropan-1-amine compound or a 2-thiazolylcyclopropan-1-amine compound.

The invention also relates to a LSD1 inhibitor (or a pharmaceutical composition comprising an LSD1 inhibitor and a pharmaceutically acceptable carrier) and one or more further therapeutic agents for use in the treatment or prevention of inflammation or an inflammatory disease or condition. In one embodiment, the inflammation or inflammatory disease or condition is chosen from atherosclerosis, respiratory distress syndrome, mesangial glomerulonephritis, chronic inflammatory bowel disease, ulcerative colitis, Crohn's disease, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, asthma, COPD, bronchial hyperresponsiveness, bronchoconstriction, airway inflammation, airway remodeling, hepatic cirrhosis, nephritis or psoriasis. In one embodiment, the further therapeutic agent is an antiinflammatory agent. In a more specific embodiment, the antiinflammatory agent is chosen from a steroid, a NSAID, or a COX-2 selective inhibitor, more preferably is chosen from a steroid, a salicylate (e.g. aspirin, diflunisal, or salsalate), a propionic acid derivative (e.g., ibuprofen, naproxen, fenoprofen, ketoprofen, flurbiprofen, oxaprozin or loxoprofen), an acetic acid derivative (e.g., indomethacin, sulindac, etodolac, ketorolac, diclofenac or nabumetone), an enolic acid derivative (e.g., piroxicam, meloxicam, tenoxicam, droxicam, lornoxicam, or isoxicam), a fenamic acid derivative (e.g., mefenamic acid, meclofenamic acid, flufenamic acid or tolfenamic acid), a selective COX-2 inhibitor (e.g., celecoxib, rofecoxib, valdecoxib, parecoxib, lumiracoxib, etoricoxib, or firocoxib), a sulphonanilide (e.g. nimesulide) or licofelone. In one embodiment, the further therapeutic agent is an antiplatelet agent. In a more specific embodiment, the antiplatelet agent is chosen from Aspirin, Clopidogrel, Prasugrel, Ticlopidine, Cilostazol, Abciximab, Eptifibatide, Tirofiban, Dipyridamole, Anagrelide, Hydroxyurea, or Epoprostenol. In one embodiment, the further therapeutic agent is an anticoagulant agent. In a more specific embodiment, the anticoagulant agent is chosen from Heparin, low molecular weight Heparins, vitamin K antagonists such as warfarin, acenocoumarol or phenprocoumon, or direct thrombin inhibitors. In one aspect, the LSD1 inhibitor is a small molecule inhibitor of LSD1. In one aspect, the LSD1 inhibitor is a selective inhibitor of LSD1. In one aspect, the LSD1 inhibitor is a selective inhibitor of LSD1 and MAOB (i.e. a dual LSD1/MAO-B inhibitor). In one aspect, the LSD1 inhibitor is a 2-cyclylcyclopropan-1-amine compound, a phenelzine compound, or a propargylamine compound, more preferably a 2-cyclylcyclopropan-1-amine compound, still more preferably a 2-arylcyclopropan-1-amine compound or a 2-heteroarylcyclopropan-1-amine compound, and even more preferably a 2-phenylcyclopropan-1-amine compound, a 2-pyridinylcyclopropan-1-amine compound or a 2-thiazolylcyclopropan-1-amine compound

### Compounds, Formulation, and Routes of Administration

In accordance with the present invention, the LSD1 inhibitor is preferably a small molecule inhibitor of LSD1. Preferably, the LSD1 inhibitor is a selective LSD1 inhibitor or a dual LSD1/MAO-B inhibitor. The LSD1 inhibitors, selective LSD1 inhibitors and dual LSD1/MAO-B inhibitors for use in the invention can be synthesized by a number of techniques including the ones that are described below.

Examples of selective LSD1 and LSD1/MAOB dual inhibitors based on a cyclylcyclopropylamine scaffold, such as arylcyclopropylamine or heteroarylcyclopropylamine are given in, e.g., WO2010/043721 (PCT/EP2009/063685), WO2010/084160 (PCT/EP2010/050697), WO2011/035941 (PCT/EP2010/055131), WO2011/042217 (PCT/EP2010/055103), WO2011/131697 (PCT/EP2011/056279), WO2012/013727 (PCT/EP2011/062947), WO2012/013728 (PCT/EP2011/062949), WO2012/045883 (PCT/EP2011/067608) and EP applications number EP10171345 (EP10171345.1), EP10187039 (EP10187039.2) and EP10171342 (EP10171342.8), all of which are all explicitly incorporated herein by reference in their entireties to the extent they are not inconsistent with the instant disclosure.

In one specific aspect, a phenylcyclopropylamine derivative or analog for use in the invention is phenylcyclopropylamine (PCPA) with one or two substitutions on the amine group; phenylcyclopropylamine with zero, one or two substitutions on the amine group and one, two three, four, or five substitution on the phenyl group; phenylcyclopropylamine with one, two, three, four, or five substitution on the phenyl group; phenylcyclopropylamine with zero, one or two substitutions on the amine group wherein the phenyl group of PCPA is substituted with (exchanged for) another ring system chosen from aryl or heterocyclyl or heteroaryl to give an aryl- or heterocyclyl- or heteroaryl-cyclopropylamine having zero, one or two substituents on the amine group; phenylcyclopropylamine wherein the phenyl group of PCPA is substituted with (exchanged for) another ring system chosen from aryl or heterocyclyl to give an aryl- or heterocycyl-cyclopropylamine wherein the aryl- or heterocyclyl-cyclopropylamine on the aryl or heterocyclyl moiety has zero, one or two substitutions on the amine group and one, two, three, four, or five substitution on the phenyl group; phenylcyclopropylamine with one, two, three, four, or five substitution on the phenyl group; or any of the above described phenylcyclopropylamine analogs or derivatives wherein the cyclo propyl has one two three or four additional substituents. Preferably, the heterocyclyl group described above in this paragraph is a heteroaryl.

Other examples of arylcyclopropylamine derivatives and analogues as LSD1 inhibitors and, accordingly, for use in the invention include tranylcypromine (Parnate™) and those disclosed in WO2010/143582 (PCT/JP2010/059476), US 2010/0324147 (U.S. Ser. No. 12/792,316), S. Mimasu et al., Biochemistry (2010), 49(30):6494-503, C. Binda et al, J Am. Chem. Soc. (2010), 132(19):6827-33, D M Gooden et al., Bioorg. Med. Chem. Let. (2008), 18:3047-3051, R Ueda et al., J. Am. Chem Soc. (2009), 131(48):17536-17537, and WO2011/131576, all of which are explicitly incorporated herein by reference in their entireties to the extent they are not inconsistent with the instant disclosure.

Other examples of LSD1 inhibitors are, e.g., phenelzine or pargyline (propargylamine) or a derivative or analog thereof. Derivatives and analogs of phenelzine and pargyline (propargylamine) include, but are not limited to, compounds where the phenyl group of the parent compound is replaced with a heteroaryl or optionally substituted cyclic group or the phenyl group of the parent compound is optionally substituted with a cyclic group and have the selective LSD1 or dual LSD1/MAO-B inhibitory activity as described herein. In one aspect, the phenelzine derivative or analog has one, two, three, four or five substituents on the phenyl group. In one aspect, the phenelzine derivative or analog has the phenyl group substituted with (exchanged for) an aryl or heterocyclyl group wherein the aryl or heterocyclyl group has zero, one, two, three, four or five substituents. In one aspect, the pargyline derivative or analog has one, two, three, four or five substituents on the phenyl group. In one aspect, the pargyline derivative or analog has the phenyl group substituted with (exchanged for) an aryl or heterocyclyl group wherein the aryl or heterocyclyl group has zero, one, two, three, four or five substituents. Methods of preparing such compounds are known to the skilled artisan.

Other LSD1 inhibitors for use in the invention include, but are not limited to bis-urea and bis-thiourea derivatives, polyamines, and guanidine/bisguanidine derivatives, such as those e.g. disclosed in S K Sharma et al. (2010) J. Med. Chem. 53 (14):5197-5212, WO 2011/022489, WO 2008/127734, WO 2007/021839, Huang et al Clinical Cancer Res 2009 15(23) 7217-28, and Huang et al Proc Nat Acad Sci USA, 2007 104(19) 8023-28, all of which are explicitly incorporated herein by reference in their entireties to the extent they are not inconsistent with the instant disclosure.

Other phenylcyclopropylamine derivatives and analogs are found, e.g., in Kaiser et al. ((1962) J. Med. Chem. 5:1243-1265); Zirkle et al. ((1962) J. Med. Chem. 1265-1284); U.S. Pat. Nos. 3,365,458; 3,471,522; 3,532,749) and Bolesov et al. ((1974) Zhurnal Organicheskoi Khimii 10:8 1661-1669) and Russian Patent No. 230169 (19681030).

The LSD1 inhibitor to be used in accordance with the present invention (e.g., in the treatment or prevention of inflammation or an inflammatory disease or condition) is preferably a 2-cyclylcyclopropan-1-amine compound, a phenelzine compound or a propargylamine compound, and is more preferably a 2-cyclylcyclopropan-1-amine compound. Said 2-cyclylcyclopropan-1-amine compound is preferably a 2-arylcyclopropan-1-amine compound or a 2-heteroarylcyclopropan-1-amine compound, more preferably a 2-phenylcyclopropan-1-amine compound, a 2-pyridinylcyclopropan-1-amine compound or a 2-thiazolylcyclopropan-1-amine compound.

It is particularly preferred that the LSD1 inhibitor or selective LSD1 inhibitor or dual LSD1/MAO-B inhibitor is a 2-cyclylcyclopropan-1-amine compound which is a compound of the following formula (I) or an enantiomer, a diastereomer or a mixture of stereoisomers (such as a racemic mixture or a diastereomer mixture) thereof, or a pharmaceutically acceptable salt or solvate thereof:

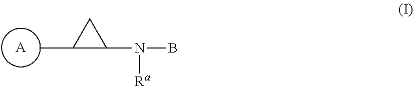

A is cyclyl optionally having 1, 2, 3 or 4 substituents A′. Preferably, said cyclyl is aryl or heteroaryl. Said aryl is preferably phenyl. Said heteroaryl is preferably selected from pyridinyl, pyrimidinyl, thiophenyl, benzothiophenyl, pyrrolyl, indolyl, furanyl or thiazolyl, more preferably said heteroaryl is selected from pyridinyl, pyrimidinyl or thiazolyl, still more preferably said heteroaryl is pyridinyl (in particular, pyridin-2-yl or pyridin-3-yl) or thiazolyl (in particular thiazol-5-yl) and even more preferably said heteroaryl is pyridin-3-yl or thiazol-5-yl.

It is preferred that said cyclyl (or said aryl or said heteroaryl, or any of the above-mentioned specific aryl or heteroaryl groups) is unsubstituted or has 1 or 2 substituents A′, and it is more preferred that said cyclyl (or said aryl or said heteroaryl, or any of the above-mentioned specific aryl or heteroaryl groups) is unsubstituted or has 1 substituent A′.

Said substituent(s) A′ is/are each independently selected from -L¹-cyclyl (e.g., -L¹-aryl, -L¹-cycloalkyl or -L¹-heterocyclyl), alkyl, alkenyl, alkynyl, alkoxy, amino, amido (e.g., —CO—NH₂), —CH₂—CO—NH₂, alkylamino, hydroxyl, nitro, halo, haloalkyl, haloalkoxy, cyano, sulfonyl, sulfinyl, sulfonamide, acyl, carboxyl, carbamate or urea, wherein the cyclyl moiety comprised in said -L¹-cyclyl is optionally further substituted with one or more (e.g., 1, 2 or 3) groups independently selected from halo, haloalkyl, haloalkoxy, aryl, arylalkoxy, aryloxy, arylalkyl, alkyl, alkenyl, alkynyl, alkoxy, amino, amido (e.g., —CO—NH₂), alkylamino, hydroxyl, nitro, —CH₂—CO—NH₂, heteroaryl, heteroarylalkoxy, heteroaryloxy, heteroarylalkyl, cyano, sulfonyl, sulfinyl, sulfonamide, acyl, carboxyl, carbamate or urea, preferably selected from halo, haloalkyl, hydroxy, N-sulfonamido or cyano. It is preferred that the cyclyl moiety comprised in said -L¹-cyclyl is unsubstituted or is substituted with one of the above groups (including, e.g., one of the preferred groups halo, haloalkyl, hydroxy, N-sulfonamido or cyano). In one preferred embodiment, the cyclyl moiety comprised in said -L¹-cyclyl is substituted with one of the above groups (including, e.g., one of the preferred groups halo, haloalkyl, hydroxy, N-sulfonamido or cyano). In another preferred embodiment the cyclyl moiety is unsubstituted. Said -L¹-cyclyl is preferably -L¹-aryl, -L¹-cycloalkyl or -L¹-heterocyclyl (e.g., -L¹-heteroaryl or -L¹-heterocycloalkyl), more preferably -L¹-aryl or -L¹-heteroaryl, even more preferably -L¹-aryl, even more preferably -L¹-phenyl.

Each L¹ is independently selected from a covalent bond, —(CH₂)₁₋₆—, —(CH₂)₀₋₃—O—(CH₂)₀₋₃—, —(CH₂)₀₋₃—NH—(CH₂)₀₋₃— or —(CH₂)₀₋₃—S—(CH₂)₀₋₃—, preferably from a covalent bond, —(CH₂)₁₋₃—, —O—(CH₂)₀₋₃— or —NH—(CH₂)₀₋₃—, more preferably from a covalent bond, —CH₂—, —O—, —O—CH₂—, —O—(CH₂)₂—, —NH— or —NH—CH₂—, even more preferably from a covalent bond, —CH₂— or —O—CH₂—. It is furthermore preferred that the aforementioned groups L¹ (connecting the moiety A to the cyclyl moiety comprised in -L¹-cyclyl) are in the specific orientation indicated above (accordingly, the group “—O—CH₂—” as an example for L¹ is preferably in the orientation ( . . . )-A-O—CH₂-cyclyl).

Preferably, said substituent(s) A′ is/are each independently selected from -L¹-aryl, -L¹-cycloalkyl, -L¹-heteroaryl or -L¹-heterocycloalkyl, wherein said aryl, said cycloalkyl, said heteroaryl or said heterocycloalkyl is optionally substituted with halo (e.g., —F or —Cl), haloalkyl (e.g., —CF₃), hydroxy, N-sulfonamido (e.g. —NHSO₂-aryl, wherein the aryl group can be optionally substituted) or cyano. More preferably, said substituent(s) A′ is/are each independently -L¹-aryl (e.g., -L¹-phenyl), wherein the aryl moiety in said -L¹-aryl (or the phenyl moiety in said -L¹-phenyl) is optionally substituted with halo (e.g., —F or —CI), haloalkyl (e.g., —CF₃), hydroxy, N-sulfonamido (e.g. —NHSO₂-aryl, wherein the aryl group can be optionally substituted) or cyano. Even more preferably, said substituent(s) A′ is/are each independently phenyl, —CH₂-phenyl, —O—CH₂-phenyl, —NH—CH₂-phenyl or —O—(CH₂)₂-phenyl, wherein said phenyl or the phenyl moiety in said —CH₂-phenyl, said —O—CH₂-phenyl, said —NH—CH₂-phenyl or said —O—(CH₂)₂-phenyl is optionally substituted with halo (e.g., —F or —Cl), haloalkyl (e.g., —CF₃), hydroxy, N-sulfonamido (e.g. —NHSO₂-aryl, wherein the aryl group can be optionally substituted) or cyano. Even more preferably, said substituent(s) A′ is/are each independently phenyl, —CH₂-phenyl, —O—CH₂-phenyl, or —O—(CH₂)₂-phenyl, wherein said phenyl or the phenyl moiety in said —CH₂-phenyl, said —O—CH₂-phenyl or said —O—(CH₂)₂-phenyl is optionally substituted with halo (e.g., —F or —Cl), haloalkyl (e.g., —CF₃), hydroxy, N-sulfonamido (e.g. —NHSO₂-aryl, wherein the aryl group can be optionally substituted) or cyano. Even more preferably, said substituent(s) A′ is/are each independently phenyl, —CH₂-phenyl, or —O—CH₂-phenyl, wherein said phenyl or the phenyl moiety in said —CH₂-phenyl or said —O—CH₂-phenyl is optionally substituted with halo (e.g., —F or —Cl) or haloalkyl (e.g., —CF₃).

It is particularly preferred that A is aryl (preferably phenyl) or heteroaryl (preferably pyridinyl or thiazolyl), which aryl or heteroaryl optionally has one substituent A′ selected from -L¹-aryl, -L¹-cycloalkyl, -L¹-heteroaryl or -L¹-heterocycloalkyl (wherein the aryl moiety in said -L¹-aryl, the cycloalkyl moiety in said -L¹-cycloalkyl, the heteroaryl moiety in said -L¹-heteroaryl or the heterocycloalkyl moiety in said -L¹-heterocycloalkyl may be substituted with halo (e.g., —F or —Cl), haloalkyl (e.g., —CF₃), hydroxy, N-sulfonamido or cyano), preferably selected from phenyl, —CH₂-phenyl or —O—CH₂-phenyl (wherein said phenyl, the phenyl moiety in said —CH₂-phenyl or the phenyl moiety in said —O—CH₂-phenyl may be substituted with halo (e.g., —F or —Cl), haloalkyl (e.g., —CF₃)), hydroxy, N-sulfonamido or cyano) and even more preferably selected from phenyl, —CH₂-phenyl or —O—CH₂-phenyl (wherein said phenyl, the phenyl moiety in said —CH₂-phenyl or the phenyl moiety in said —O—CH₂-phenyl may be substituted with halo (e.g., —F or —Cl) or haloalkyl (e.g., —CF₃)).

R^{a} is —H or alkyl. Preferably R^{a} is —H or (C1-C4)alkyl (such as methyl or ethyl), and more preferably R^{a} is —H.

B is -L²-cyclyl, —H, -L²-CO—NH₂, -L²-CO—NR¹R², or -L²-CO—R³, wherein the cyclyl moiety in said -L²-cyclyl is optionally substituted with one or more (e.g., one, two or three) groups independently selected from halo, haloalkyl, haloalkoxy, haloaryl, aryl, arylalkoxy, aryloxy, arylalkyl, alkyl, alkenyl, alkynyl, alkoxy, amino, amido (e.g., —CO—NH₂), alkylamino, hydroxyl, nitro, —CH₂—CO—NH₂, heteroaryl, heteroarylalkoxy, heteroaryloxy, heteroaryl alkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea, preferably selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide.

It is preferred that the cyclyl moiety in said -L²-cyclyl is unsubstituted or is substituted with one group selected from halo, haloalkyl, haloalkoxy, haloaryl, aryl, arylalkoxy, aryloxy, arylalkyl, alkyl, alkenyl, alkynyl, alkoxy, amino, amido (e.g., —CO—NH₂), alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea, preferably selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide.

The cyclyl moiety in said -L²-cyclyl, which may be substituted as defined and described above, is preferably selected from aryl, cycloalkyl or heterocyclyl (e.g., heteroaryl or heterocycloalkyl), more preferably heterocyclyl, even more preferably from heteroaryl or heterocycloalkyl. Said heteroaryl is preferably selected from oxadiazolyl, thiazolyl or pyrimidinyl. Said heterocycloalkyl is preferably selected from pyrrolidinyl, piperidinyl, piperazinyl, N-methylpiperazinyl or morpholinyl.

In formula (I), R¹ and R² are each independently chosen from H, alkyl, alkynyl, alkenyl, -L-carbocyclyl, -L-aryl, or -L-heterocyclyl, wherein said alkyl, said alkynyl or said alkenyl is optionally substituted with one or more groups independently selected from halo, haloalkoxy, haloaryl, aryl, arylalkoxy, aryloxy, alkoxy, amino, amido, alkylamino, hydroxyl, nitro, —CH₂—CO—NH₂, heteroaryl, heteroarylalkoxy, heteroaryloxy, cycloalkyl, cycloalkylalkoxy, cycloalkoxy, heterocycloalkyl, heterocycloalkylalkoxy, heterocycloalkoxy, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea, and further wherein the carbocyclyl moiety in said -L-carbocyclyl, the aryl moiety in said -L-aryl, or the heterocyclyl moiety in said -L-heterocyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea.

In formula (I), R³ is chosen from -L-heterocyclyl, -L-carbocyclyl, -L-aryl, —H, or alkoxy, wherein the carbocyclyl moiety in said -L-carbocyclyl, the heterocyclyl moiety in said -L-heterocyclyl or the aryl moiety in said -L-aryl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea. It is preferred that R³ is -L-heterocyclyl, particularly -L-heterocyclyl wherein the heterocyclyl moiety is a saturated heterocyclic ring, and more preferably it is preferred that L is a covalent bond.

Each L is independently selected from —(CH₂)—(CH₂)ₙ—, —(CH₂)C(═O)(CH₂)ₙ—, —(CH₂)ₙC(═O)NH(CH₂)ₙ—, —(CH₂)ₙNHC(═O)O(CH₂)ₙ—, —(CH₂)ₙNHC(═O)NH(CH₂)—, —(CH₂)NHC(═S)S(CH₂)ₙ—, —(CH₂)ₙOC(═O)S(CH₂)ₙ—, —(CH₂)ₙNH(CH₂)ₙ—, —(CH₂)ₙO(CH₂)ₙ—, —(CH₂)ₙS(CH₂)ₙ—, and (CH₂)ₙNHC(═S)NH(CH₂)ₙ—, and each n is independently chosen from 0, 1, 2, 3, 4, 5, 6, 7, and 8. Preferably, in R¹ and R² each L is independently —(CH₂)₁₋₆—, more preferably —(CH₂)₁₋₄, and even more preferably —CH₂—. Preferably, in R³ L is bond.

L² is C₁₋₁₂ alkylene which is optionally interrupted by one or more (e.g., one, two, three or four) groups independently selected from —O—, —S—, —NH—, —N(alkyl)-, —CO—, —CO—NH— or —CO—N(alkyl)-, or L² is a covalent bond. Preferably, L² is —CH₂—(C₁₋₆ alkylene), —CH₂—CO— or a covalent bond, wherein the alkylene moiety in said —CH₂—(C₁₋₆ alkylene) is optionally interrupted by one or more (e.g., one, two or three) groups independently selected from —O—, —S—, —NH—, -(alkyl)-, —CO—, —CO—NH—, —CO—N(alkyl)-. More preferably, L² is —(CH₂)₁₋₄—, —CH₂—CO— or a covalent bond. Even more preferably, L² is —CH₂—, —(CH₂)₂—, —CH₂—CO— or a covalent bond.

In one preferred embodiment, B is -L²-cyclyl, wherein the cyclyl moiety in said -L²-cyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea.

In another preferred embodiment, B is —(CH₂)₀₋₅-heteroaryl, —(CH₂)₀₋₅-heterocycloalkyl, —(CH₂)₁₋₅—CO-heterocycloalkyl, —H, —(CH₂)₁₋₄—CO—NH₂, or —(CH₂)₁₋₄—CO—NR¹R², wherein the heteroaryl moiety comprised in said —(CH₂)₀₋₅-heteroaryl and the heterocycloalkyl moiety comprised in said —(CH₂)₀₋₅-heterocycloalkyl or in said —(CH₂)₁₋₅—CO-heterocycloalkyl is optionally substituted with one or two groups, preferably with one group, independently selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide.

In a particularly preferred embodiment, B is —(CH₂)₀₋₅-heteroaryl, wherein the heteroaryl moiety comprised in said —(CH₂)₀₋₅-heteroaryl is preferably selected from oxadiazolyl, thiazolyl or pyrimidinyl and, furthermore, is optionally substituted with one group selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide. In a further particularly preferred embodiment, B is —(CH₂)₀₋₅-heterocycloalkyl, wherein the heterocycloalkyl moiety comprised in said —(CH₂)₀₋₅-heterocycloalkyl is preferably selected from pyrrolidinyl, piperidinyl, piperazinyl, N-methylpiperazinyl or morpholinyl and, furthermore, is optionally substituted with one group selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide. In a further particularly preferred embodiment, B is —CH₂-oxadiazolyl, wherein the oxadiazolyl moiety comprised in said —CH₂-oxadiazolyl is optionally substituted with one group selected from halo, alkyl, alkoxy, haloalkyl, haloalkoxy, cyano, hydroxyl, amino, alkylamino or aminoalkyl (accordingly, B may, for example, be aminooxadiazolylmethyl, such as 2-amino-1,3,4-oxadiazol-5-ylmethyl or 3-amino-1,2,4-oxadiazol-5-ylmethyl). In a further particularly preferred embodiment, B is —(CH₂)₁₋₅—CO-heterocycloalkyl, wherein the heterocycloalkyl moiety comprised in said —(CH₂)₁₋₅—CO-heterocycloalkyl is preferably selected from pyrrolidinyl, piperidinyl, piperazinyl, N-methylpiperazinyl or morpholinyl and, furthermore, is optionally substituted with one group selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide. In a further particularly preferred embodiment, B is —H. In a further particularly preferred embodiment, B is -L²-CO—NH₂, preferably —(CH₂)₁₋₄—CO—NH₂, more preferably —CH₂—CO—NH₂. In a further particularly preferred embodiment, B is L₂-CO—NR¹R^{2′} preferably B is —(CH₂)₁₋₄—CO—NR¹R², more preferably —CH₂—CO—NR¹R².

The substituents on the cyclopropane ring, i.e. the groups -(A) and —NR^{a}—B, are preferably in trans configuration. In that case, the 2-cyclylcyclopropan-1-amine compound of formula (I) may have the configuration (1R,2S) or the configuration (1S,2R) at the cyclopropane ring carbon atoms. The present invention specifically relates to the (1R,2S) stereoisomer of the 2-cyclylcyclopropan-1-amine compound of formula (I). The invention also specifically relates to the (1S,2R) stereoisomer of the 2-cyclylcyclopropan-1-amine compound of formula (I).

In one embodiment, the LSD1 inhibitor to be used in the present invention is a 2-cyclylcyclopropan-1-amine compound which is a compound of the following formula (II) or a pharmaceutically acceptable salt thereof:

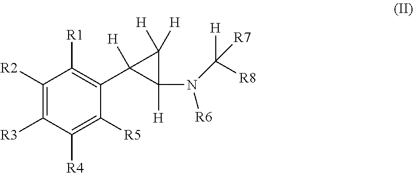

In formula (II), each of R1-R5 is optionally substituted and independently chosen from —H, halo, alkyl, alkoxy, cycloalkoxy, haloalkyl, haloalkoxy, -L-aryl, -L-heteroaryl, -L-heterocyclyl, -L-carbocycle, acylamino, acyloxy, alkylthio, cycloalkylthio, alkynyl, amino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;
R6 is chosen from —H and alkyl;
R7 is chosen from —H, alkyl, and cycloalkyl;
R8 is chosen from C(═O)NRₓR_{y} and —C(═O)R_{z};
Rₓ when present is chosen from H, alkyl, alkynyl, alkenyl, -L-carbocycle, -L-aryl, -L-heterocyclyl, all of which are optionally substituted;
R_{y} when present is chosen from H, alkyl, alkynyl, alkenyl, -L-carbocycle, -L-aryl, -L-heterocyclyl, all of which are optionally substituted;
R_{z} when present is chosen from H, alkoxy, -L-carbocyclic, -L-heterocyclic, -L-aryl, wherein the aryl, heterocyclyl, or carbocycle is optionally substituted;
each L can be saturated, partially saturated, or unsaturated, and is independently chosen from —(CH₂)ₙ—(CH₂)—, —(CH₂)ₙC(═O)(CH₂)ₙ—, —(CH₂)C(═O)NH(CH₂)ₙ—, —(CH₂)ₙNHC(═O)O(CH₂)ₙ—, —(CH₂)ₙNHC(═O)NH(CH₂)₁₁—, (CH₂)ₙNHC(═S)S(CH₂)ₙ—, —(CH₂)ₙOC(═O)S(CH₂)ₙ—, —(CH₂)ₙNH(CH₂)ₙ—, —(CH₂)ₙO(CH₂)ₙ—, —(CH₂)ₙS(CH₂)ₙ—, and —(CH₂)ₙNHC(═S)NH(CH₂)ₙ—, where each n is independently chosen from 0, 1, 2, 3, 4, 5, 6, 7, and 8, wherein optionally substituted refers to zero or 1 to 4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, heteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido.

In a further embodiment, the LSD1 inhibitor to be used in the present invention is a 2-cyclylcyclopropan-1-amine compound which is a compound of the following formula (III) or a pharmaceutically acceptable salt thereof:

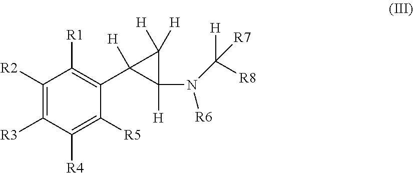

In formula (III), each of R1-R5 is independently chosen from —H, halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido;
R6 is chosen from —H and alkyl;
R7 is chosen from —H, alkyl, and cycloalkyl;
R8 is a -L-heterocyclyl wherein the ring or ring system of said -L-heterocyclyl has from 0-3 substituents chosen from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido; or
R8 is -L-aryl wherein the ring or ring system of said -L-aryl has from 1-3 substituents chosen from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido;
each L is independently chosen from —(CH₂)ₙ—(CH₂)ₙ—, —(CH₂)NH(CH₂)ₙ—, —(CH₂)ₙO(CH₂)—, and —(CH₂)ₙS(CH₂)ₙ—, and where each n is independently chosen from 0, 1, 2, and 3.

In a further embodiment, the LSD1 inhibitor to be used in the present invention is a 2-cyclylcyclopropan-1-amine compound which is a compound of the following formula (IV) or an enantiomer, diastereomer, or mixture thereof, or a pharmaceutically acceptable salt or solvate thereof:

(A′)_{X}-(A)-(B)—(Z)-(L)-(D) (IV)

In formula (IV), (A) is heteroaryl or aryl;
each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2, or 3 substituents independently chosen from halo, haloalkyl, aryl, arylalkoxy, alkyl, alkoxy, cyano, sulfonyl, amido, and sulfinyl;
X is 0, 1, 2, or 3;
(B) is a cyclopropyl ring, wherein (A) and (Z) are covalently bonded to different carbon atoms of (B);
(Z) is —NH—;
(L) is chosen from —CH₂CH₂—, —CH₂CH₂CH₂—, and —CH₂CH₂CH₂CH₂—; and
(D) is chosen from —N(—R1)-R2, —O—R3, and —S—R3, wherein:
R1 and R2 are mutually linked to form a heterocyclic ring together with the nitrogen atom that R1 and R2 are attached to, wherein said heterocyclic ring has 0, 1, 2, or 3 substituents independently chosen from —NH₂, —NH(C₁-C₆ alkyl), —N(C₁-C₆ alkyl)(C₁-C₆ alkyl), alkyl, halo, cyano, alkoxy, haloalkyl, and haloalkoxy, or
R1 and R2 are independently chosen from —H, alkyl, cycloalkyl, haloalkyl, and heterocyclyl, wherein the sum of substituents on R1 and R2 together is 0, 1, 2, or 3, and the substituents are independently chosen from —NH₂, —NH(C₁-C₆ alkyl), —N(C₁-C₆ alkyl)(C₁-C₆ alkyl), and fluoro; and
R3 is chosen from —H, alkyl, cycloalkyl, haloalkyl, and heterocyclyl, wherein
R3 has 0, 1, 2, or 3 substituents independently chosen from —NH₂, —NH(C₁-C₆ alkyl), —N(C₁-C₆ alkyl)(C₁-C₆ alkyl), and fluoro;
with the proviso that the following compounds are excluded:
N1-[(trans)-2-phenylcyclopropyl]-N2-undecyl-rel-1,2-ethanediamine;
N1-[(trans)-2-phenylcyclopropyl]-N2-tricyclo[3,3,1,13,7]dec-2-yl-rel-1,2-ethanediamine;
N1-cyclooctyl-N2-[(trans)-2-phenylcyclopropyl]-rel-1,2-ethanediamine;
N1,N1-dimethyl-N2-(2-phenylcyclopropyl)-1,3-propanediamine;
N1,N1-dimethyl-N2-(2-phenylcyclopropyl)-1,2-ethanediamine; and
trans-1-phenyl-2-[(2-hydroxyethyl)amino]cyclopropane.

In a further embodiment, the LSD1 inhibitor to be used in the present invention is a 2-cyclylcyclopropan-1-amine compound which is a compound of the following formula (V) or a pharmaceutically acceptable salt or solvate thereof:

(A′)_{X}-(A)-(B)—(Z)-(L)-C(═O)NH₂ (V)

In formula (V), (A) is heteroaryl or aryl;
each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2 or 3 substituents independently chosen from halo, haloalkyl, aryl, arylalkoxy, alkyl, alkoxy, cyano, sulfonyl, sulfinyl, and carboxamide;
X is 0, 1, 2, or 3;
(B) is a cyclopropyl ring, wherein (A) and (Z) are covalently bonded to different carbon atoms of (B);
(Z) is —NH—; and
(L) is —(CH₂)ₘCR₁R₂—, wherein m is 0, 1, 2, 3, 4, 5, or 6, and wherein R₁ and R₂ are each independently hydrogen or C₁-C₆ alkyl;
provided that, if (L) is —CH₂— or —CH(CH₃)—, then X is not 0.

In a further embodiment, the LSD1 inhibitor to be used in the present invention is a 2-cyclylcyclopropan-1-amine compound which is a compound of the following formula (VI) or an enantiomer, a diastereomer, or a mixture thereof, or a pharmaceutically acceptable salt or solvate thereof:

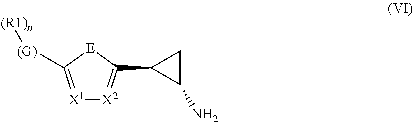

In formula (VI), E is —N(R3)-, —O—, or S—, or is —X³═X⁴—;
X¹ and X² are independently C(R2) or N;
X³ and X⁴, when present, are independently C(R2) or N;
(G) is a cyclyl group;
each (R1) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;
each (R2) is independently chosen from H, alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 1, 2, or 3 independently chosen optional substituents or two (R2) groups can be taken together to form a heterocyclyl or aryl group having 1, 2, or 3 independently chosen optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate;
R3 is —H or a (C₁-C₆)alkyl group;
each L1 is independently alkylene or heteroalkylene; and
n is 0, 1, 2, 3, 4 or 5.

In a further embodiment, the LSD1 inhibitor to be used in the present invention is a 2-cyclylcyclopropan-1-amine compound which is a compound of the following formula (VII) or an enantiomer, a diastereomer, or a mixture thereof, or a pharmaceutically acceptable salt or solvate thereof:

(A′)_{X}-(A)-(B)—(Z)-(L)-(D) (VII)

In formula (VII), (A) is heteroaryl or aryl;
each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2, or 3 substituents independently chosen from halo, haloalkyl, haloalkoxy, aryl, arylalkoxy, alkyl, alkoxy, amido, —CH₂C(═O)NH₂, heteroaryl, cyano, sulfonyl, and sulfinyl;
X is 0, 1, 2, or 3;
(B) is a cyclopropyl ring, wherein (A) and (Z) are covalently bonded to different carbon atoms of (B);
(Z) is —NH—;
(L) is chosen from a single bond, —CH₂—, —CH₂CH₂—, —CH₂CH₂CH₂—, and —CH₂CH₂CH₂CH₂—; and
(D) is an aliphatic carbocyclic group or benzocycloalkyl, wherein said aliphatic carbocyclic group or said benzocycloalkyl has 0, 1, 2, or 3 substituents independently chosen from —NH₂, —NH(C₁-C₆ alkyl), —N(C₁-C₆ alkyl)(C₁-C₆ alkyl), alkyl, halo, amido, cyano, alkoxy, haloalkyl, and haloalkoxy;
with the proviso that the following compounds are excluded:
N-(2-phenylcyclopropyl)-cyclopentanamine;
10,11-dihydro-N-(2-phenylcyclopropyl)-5H-dibenzo[a,d]cyclohepten-5-amine;
and
trans-N-(2-phenylcyclopropyl)-cyclohexanamine.

In a further embodiment, the LSD1 inhibitor to be used in the present invention is a 2-cyclylcyclopropan-1-amine compound which is a compound of the following formula (VIII) or a pharmaceutically acceptable salt or solvate thereof:

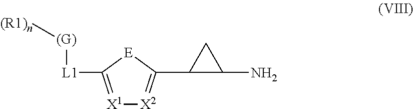

In formula (VIII), E is —X³═X⁴—, —N(R3)-, —S—, or —O—;
X¹ and X² are each independently C(R2) or N;
X³ and X⁴, when present, are each independently C(R2) or N;
L1 is —NH— or —NH—CH₂—;
G is a cyclyl group;
each R1 is independently chosen from alkyl, alkenyl, alkynyl, cyclyl, -L2-cyclyl, -L2-amino, -L2-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;
each R2 is independently chosen from H, alkyl, alkenyl, alkynyl, cyclyl, -L2-cyclyl, -L2-amino, -L2-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each R2 group has 1, 2, or 3 independently chosen optional substituents, and further wherein two R2 groups bound to adjacent carbon atoms can be taken together to form a heterocyclyl or aryl group having 1, 2, or 3 independently chosen optional substituents; wherein said optional substituents are each independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfinyl, sulfonyl, sulfonamide, urea or carbamate;
R3 is —H or an (C1-C6)alkyl group;
each L2 is independently chosen from alkylene or heteroalkylene; and
n is 0, 1, 2, 3, 4 or 5.

In a further embodiment, the LSD1 inhibitor to be used in the present invention is a 2-cyclylcyclopropan-1-amine compound which is a compound of the following formula (IX) or a pharmaceutically acceptable salt or solvate thereof:

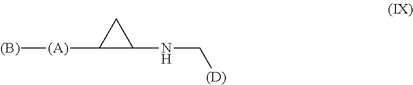

In formula (IX), (A) is a cyclyl group having n substituents (R3);
(B) is a cyclyl group or an -(L1)-cyclyl group, wherein said cyclyl group or the cyclyl moiety comprised in said -(L1)-cyclyl group has n substituents (R2);
(L1) is —O—, —NH—, —N(alkyl)-, alkylene or heteroalkylene;
(D) is a heteroaryl group or an -(L2)-heteroaryl group, wherein said heteroaryl group or the heteroaryl moiety comprised in said -(L2)-heteroaryl group has one substituent (R1), and further wherein said heteroaryl group is covalently bonded to the remainder of the molecule through a ring carbon atom or the heteroaryl moiety comprised in said -(L2)-heteroaryl group is covalently bonded to the
(L2) moiety through a ring carbon atom;
(L2) is —O—, —NH—, —N(alkyl)-, alkylene or heteroalkylene;
(R1) is a hydrogen bonding group such as, e.g., —OH, —NH₂, amido, —S(O)₂NH₂, —C(═O)NH₂, —CH₂—C(═O)NH₂, —NH—C(═O)CH₃, —NHCH₃, —N(CH₃)₂ or —CH₂—NH₂;
each (R2) is independently selected from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate or urea;
each (R3) is independently selected from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate, or urea; and
n is independently 0, 1, 2, 3 or 4.

Exemplary non-limiting selective LSD1 inhibitors are OG Compounds A, B, C and D as shown in FIG. 1 and Compounds 3, 4 and 6 to 9 as shown in Example 2, as well as pharmaceutically acceptable salts or solvates thereof. Exemplary non-limiting dual LSD1/MAO-B selective inhibitors are OG Compounds E and F as shown in FIG. 2 and Compounds 1 and 2 as shown in Example 2, as well as pharmaceutically acceptable salts or solvates thereof.

In an initial determination, the IC50 values of OG Compound A were found to be <0.1 μM for LSD1, 15-20 μM for MAO-A and 1-5 μM for MAO-B, the IC50 values of OG Compound D were found to be <0.02 μM for LSD1 and 0.5-2 μM for MAO-A, the IC50 values of OG Compound E were found to be <0.5 μM for LSD1 and 10-20 μM for MAO-A, and the IC50 value of OG Compound F for MAO-A was found to be >40 μM. In a further, more elaborate determination, the IC50 values as provided in FIGS. 1 and 2 have been obtained. These values confirm that OG Compounds A to D are selective LSD1 inhibitors and OG Compounds E and F are dual LSD1/MAO-B selective inhibitors.

The 2-cyclylcyclopropan-1-amine compounds disclosed and described herein, including, e.g., the compounds of formulae (I) to (IX), can be prepared by methods known in the art of synthetic chemistry. For example, these compounds can be prepared in accordance with or in analogy to the methods described in WO2010/043721, WO2010/084160, WO2011/035941, WO2011/042217, WO2011/131697, WO2012/013727, WO2012/013728 and WO2012/045883.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

As used herein, the term “aryl,” refers a carbocyclic aromatic system containing one ring, or two or three rings fused together where in the ring atoms are all carbon. The term “aryl” group includes, but is not limited to groups such as phenyl, naphthyl, or anthracenyl. A preferred aryl group is phenyl.

As used herein, the term “heterocyclyl” or “heterocycle,” each refer to a saturated, partially unsaturated, or fully unsaturated monocyclic, bicyclic, or tricyclic heterocyclic group containing at least one heteroatom as a ring member, wherein each heteroatom may be independently selected from the group consisting of nitrogen, oxygen, and sulfur wherein the nitrogen and/or sulfur atoms may be oxidized (e.g., —N═O, —S(═O)—, or —S(═O)₂—). Additionally, 1, 2, or 3 of the carbon atoms of the heterocyclyl may be optionally oxidized (e.g., to give an oxo group or ═O). One group of heterocyclyls has from 1 to 4 heteroatoms as ring members. Another group of heterocyclyls has from 1 to 2 heteroatoms as ring members. One group of heterocyclyls has from 3 to 8 ring members in each ring. Yet another group of heterocyclyls has from 3 to 7 ring members in each ring. Again another group of heterocyclyls has from 5 to 6 ring members in each ring. “Heterocyclyl” is intended to encompass a heterocyclyl group fused to a carbocyclyl or benzo ring systems. Examples of heterocyclyl groups include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, or imidazolidinyl. Examples of heteroaryls that are heterocyclyls include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, or furopyridinyl.

As used herein, the term “heteroaryl,” refers to a 3 to 7 membered unsaturated monocyclic ring, or a fused bicyclic, or tricyclic ring system in which the rings are aromatic and in which at least one ring contains at least one atom selected from the group consisting of O, S, and N. One group of heteroaryls has from 5 to 7 ring atoms. Examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, or furopyridinyl.

As used herein, the term “acyl,” refers to a carbonyl attached to an alkenyl, alkyl, aryl, cycloalkyl, heteroaryl, heterocyclyl, or any other moiety where the atom attached to the carbonyl is carbon. An “acetyl” group refers to a —C(═O)CH₃ group. An “alkylcarbonyl” or “alkanoyl” group refers to an alkyl group attached to the parent molecular moiety through a carbonyl group. Examples of such groups include, but are not limited to, methylcarbonyl or ethylcarbonyl. Examples of acyl groups include, but are not limited to, formyl, alkanoyl or aroyl.

As used herein, the term “alkenyl,” refers to a straight-chain or branched-chain hydrocarbon group having one or more double bonds and containing from 2 to 20 carbon atoms. Exemplary alkenyl groups may have from 2 to 6 carbon atoms. A (C2-C6)alkenyl has from 2 to 6 carbon atoms.

As used herein, the term “alkoxy,” refers to an alkyl ether group, wherein the term alkyl is as defined below. Exemplary alkoxy groups may have from 1 to 6 carbon atoms. Examples of suitable alkyl ether groups include, but are not limited to, methoxy, ethoxy, n-propoxy, isopropoxy, n-butoxy, iso-butoxy, sec-butoxy, tert-butoxy, or n-pentoxy.

As used herein, the term “alkyl,” refers to a straight-chain or branched-chain alkyl group containing from 1 to 20 carbon atoms. Exemplary alkyl groups may have from 1 to 10 or, in particular, from 1 to 6 carbon atoms. A (C1-C10)alkyl has from 1 to 10 carbon atoms and a (C1-C6)alkyl has from 1 to 6 carbon atoms and a (C1-C4)alkyl has from 1 to 4 carbon atoms. Examples of alkyl groups include, but are not limited to, methyl, ethyl, n-propyl, isopropyl, n-butyl, isobutyl, sec-butyl, tert-butyl, pentyl, isopentyl, neo-pentyl, iso-amyl, hexyl, heptyl, octyl, or nonyl.

As used herein, the term “alkylene” refers to an alkyl group attached at two positions, i.e. an alkanediyl group. Exemplary alkylene groups may have from 1 to 6 carbon atoms. Examples include, but are not limited to, methylene, ethylene, propylene, butylene, pentylene, hexylene, heptylene, octylene, or nonylene.

As used herein, the term “alkylamino,” refers to an alkyl group attached to the parent molecular moiety through an amino group. Suitable alkylamino groups may be mono- or dialkylated, forming groups including, but not limited to N-methylamino, N-ethylamino, N,N-dimethylamino, N,N-ethylmethylamino, N,N-diethylamino, N-propylamino, and N,N-methylpropylamino.

As used herein, the term “alkynyl,” refers to a straight-chain or branched-chain hydrocarbon group having one or more triple bonds and containing from 2 to 20 carbon atoms. Exemplary alkynyl groups may have from 2 to 6 carbon atoms. A (C2-C6)alkynyl has from 2 to 6 carbon atoms. A (C2-C4)alkynyl has from from 2 to 4 carbon atoms. Examples of alkynyl groups include, but are not limited to, ethynyl, propynyl, hydroxypropynyl, butyn-1-yl, butyn-2-yl, pentyn-1-yl, 3-methylbutyn-1-yl, or hexyn-2-yl.

As used herein, the terms “amido” and “carbamoyl,” refer to an amino group as described below attached to the parent molecular moiety through a carbonyl group (e.g., —C(═O)NRR′), or vice versa (—N(R)C(═O)NR′). “Amido” and “carbamoyl” encompass “C-amido”, “N-amido” and “acylamino” as defined herein. R and R′ are as defined herein.

As used herein, the term “C-amido,” refers to a —C(═O)NRR′ group with R and R′ as defined herein.

As used herein, the term “amino,” refers to —NRR′, wherein R and R′ are independently selected from the group consisting of hydrogen, alkyl, heteroalkyl, aryl, carbocyclyl, and heterocyclyl. Additionally, R and R′ may be combined to form a heterocyclyl.

As used herein, the term “arylalkoxy” or “aralkoxy,” refers to an aryl group attached to the parent molecular moiety through an alkoxy group. Examples of arylalkoxy groups include, but are not limited to, benzyloxy or phenethoxy.

As used herein, the term “arylalkyl” or “aralkyl,” refers to an aryl group attached to the parent molecular moiety through an alkyl group.

As used herein, the term “aryloxy,” refers to an aryl group attached to the parent molecular moiety through an oxy (—O—).

As used herein, the term “carbamate,” refers to an O-carbamyl or N-carbamyl group as defined herein.

As used herein, the term “carbonyl,” when alone includes formyl —C(═O)H and in combination is a —C(═O)— group.

As used herein, the term “carboxyl” or “carboxy” refers to —C(═O)OH or the corresponding “carboxylate” anion, such as is in a carboxylic acid salt. An “O-carboxy” group refers to a RC(═O)O— group, where R is as defined herein. A “C-carboxy” group refers to a —C(═O)OR groups where R is as defined herein.

As used herein, the term “cyano” refers to —CN.

As used herein, the term “carbocyclyl” refers to a saturated or partially saturated monocyclic or a fused bicyclic or tricyclic group wherein the ring atoms of the cyclic system are all carbon and wherein each cyclic moiety contains from 3 to 12 carbon atom ring members. “Carbocyclyl” encompasses benzo fused to a carbocyclyl ring system. One group of carbocyclyls have from 5 to 7 carbon atoms. Examples of carbocyclyl groups include, but are not limited to, cyclopropyl, cyclobutyl, cyclopentyl, cyclohexyl, and cycloheptyl, tetrahydronapthyl, indanyl, octahydronaphthyl, 2,3-dihydro-1H-indenyl, or adamantyl.

As used herein, the term “cycloalkyl” refers to a saturated monocyclic, bicyclic or tricyclic group wherein the ring atoms of the cyclic system are all carbon and wherein each cyclic moiety contains from 3 to 12 carbon atom ring members. One group of cycloalkyls has from 5 to 7 carbon atoms. Examples of cycloalkyl groups include, but are not limited to, cyclopropyl, cyclobutyl, cyclopentyl, cyclohexyl, cycloheptyl, or adamantyl.

As used herein, the term “cycloalkenyl” refers to a partially saturated monocyclic, bicyclic or tricyclic group wherein the ring atoms of the cyclic system are all carbon and wherein each cyclic moiety contains from 3 to 12 carbon atom ring members. One group of carboalkenyls have from 5 to 7 carbon atoms. Examples of cycloalkenyl groups include, but are not limited to, cyclobutenyl, cyclopentenyl, or cyclohexenyl.

As used herein, the term “cyclyl” refers to an aryl, heterocyclyl, or carbocyclyl group as defined herein. A “cyclyl” group may, for example, be an aryl group, a cycloalkyl group, a heteroaryl group or a heterocycloalkyl group.

As used herein, the term “halo” or “halogen” refers to fluorine, chlorine, bromine, or iodine.

As used herein, the term “haloalkoxy” refers to a haloalkyl group attached to the parent molecular moiety through an oxygen atom. Examples of haloalkoxy groups include, but are not limited to, trifluoromethoxy, 2-fluoroethoxy, or 3-chloropropoxy.

As used herein, the term “haloalkyl” refers to an alkyl group having the meaning as defined above wherein one or more hydrogens are replaced with a halogen. Specifically embraced are monohaloalkyl, dihaloalkyl or polyhaloalkyl groups. A monohaloalkyl group, for one example, may have an iodo, bromo, chloro or fluoro atom within the group. Dihalo or polyhaloalkyl groups may have two or more of the same halo atoms or a combination of different halo groups. Examples of haloalkyl groups include, but are not limited to, fluoromethyl, difluoromethyl, trifluoromethyl, chloromethyl, dichloromethyl, tri chloromethyl, pentafluoroethyl, heptafluoropropyl, difluorochloromethyl, dichlorofluoromethyl, difluoroethyl, difluoropropyl, dichloroethyl or dichloropropyl.

As used herein, the term “heteroalkyl” refers to a straight or branched alkyl chain, as defined herein above (e.g., an alkyl chain having from 1 to 6 carbon atoms), wherein one, two, or three carbons forming the alkyl chain are each replaced by a heteroatom independently selected from the group consisting of O, N, and S, and wherein the nitrogen and/or sulfur heteroatom(s) (if present) may optionally be oxidized and the nitrogen heteroatom(s) (if present) may optionally be quaternized. The heteroatom(s) O, N and S may, for example, be placed at an interior position of the heteroalkyl group, i.e., the heteroalkyl may be bound to the remainder of the molecule via a carbon atom. Up to two heteroatoms may be consecutive, such as, for example, —CH₂—NH—OCH₃.

As used herein, the term “heteroalkylene” refers to a heteroalkyl group attached at two positions. Examples include, but are not limited to, —CH₂OCH₂—, —CH₂SCH₂—, and —CH₂NHCH₂—, —CH₂S—, or —CH₂NHCH(CH₃)CH₂—.

As used herein, the term “heterocycloalkyl” refers to a heterocyclyl group that is not fully unsaturated e.g., one or more of the rings systems of a heterocycloalkyl is not aromatic. Examples of heterocycloalkyls include piperazinyl, morpholinyl, piperidinyl, or pyrrolidinyl.

As used herein, the term “hydroxyl” or “hydroxy” as used herein, refers to —OH.

As used herein, the term “hydroxyalkyl” as used herein, refers to a hydroxyl group attached to the parent molecular moiety through an alkyl group.

As used herein, the phrase “in the main chain,” refers to the longest contiguous or adjacent chain of carbon atoms starting at the point of attachment of a group to the compounds of any one of the formulas disclosed herein.

As used herein, the term phrase “linear chain of atoms” refers to the longest straight chain of atoms independently selected from carbon, nitrogen, oxygen and sulfur.

As used herein, the term “lower” where not otherwise specifically defined, means containing from 1 to and including 6 carbon atoms.

As used herein, the term “lower aryl” means phenyl or naphthyl.

As used herein, the term “lower heteroaryl” means monocyclic heteroaryl comprising five or six ring members, of which between one and four said members may be heteroatoms selected from O, S, or N.

As used herein, the terms “benzo” and “benz” refer to the divalent group C₆H₄═ derived from benzene. Examples include, but are not limited to, benzothiophene or benzimidazole.

As used herein, the term “nitro” refers to —NO₂.

As used herein, the terms “sulfonate” “sulfonic acid” and “sulfonic” refers to the —SO₃H group and its anion as the sulfonic acid is used in salt formation.

As used herein, the term “sulfanyl” refers to —S—.

As used herein, the term “sulfinyl” refers to —S(═O)R, with R as defined herein.

As used herein, the term “sulfonyl” refers to —S(═O)₂R, with R as defined herein.

As used herein, the term “sulfonamide” refers to an N-sulfonamido or S-sulfonamido group as defined herein. As used herein, the term “N-sulfonamido” refers to a RS(═O)₂N(R′)— group with R and R′ as defined herein. Exemplary, non-limiting N-sulfonamido groups are NHSO₂ alkyl such as NHSO₂CH₃, —NHSO₂CH₂CH₃ or —NHSO₂ (isopropyl), and NHSO₂ (optionally substituted aryl) such as —NHSO₂ phenyl. As used herein, the term “S-sulfonamido” refers to a —S(═O)₂NRR′, group, with R and R′ as defined herein.

As used herein, the term “urea” refers to a N(R)C(═O)N(R)(R′) group wherein each R and R′ independently are as defined herein.

As used herein, “hydrogen bonding group” refers to a substituent group, which is capable of taking part in a non-covalent bonding between hydrogen and another atom (usually nitrogen or oxygen). Examples include, but are not limited to, —OH, NH₂, —OH, amido, —S(O)₂NH₂, —C(═O)NH₂, —CH₂—C(═O)NH₂, —NH—C(═O)CH₃, —NHCH₃, —N(CH₃)₂ and —CH₂—NH₂.

As used herein, the term “optionally substituted” means the preceding or anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower cycloalkyl, phenyl, aryl, acyloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxyl, amino, lower alkylamino, arylamino, aminoalkyl, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, carbamate, and urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.” In one specific definition, the optional substituents are chosen from hydroxyl, halo, alkyl, alkoxy, haloalkyl, haloalkoxy, —N((C1-C3)alkyl)₂, —NH((C1-C3)alkyl), —NHC(═O)((C1-C3)alkyl), —C(═O)OH, —C(═O)O((C1-C3)alkyl), —C(═O)(C1-C3)alkyl), —C(═O)NH₂, —C(═O)NH((C1-C3)alkyl), —C(═O)NH(cycloalkyl), C(═O)N((C1-C3)alkyl)₂, —S(═O)₂((C1-C3)alkyl), —S(═O)₂NH₂, —S(═O)₂N((C1-C3)alkyl)₂, —S(═O)₂NH((C1-C3)alkyl), —CHF₂, —OCF₃, —OCHF₂, —SCF₃, —CF₃, —CN, —NH₂, —NO₂, or tetrazolyl.

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl.

Whether an R group has a number designation or not, every R group, including R, R′ and RP where p=(1, 2, 3, . . . p), every substituent, and every term should be understood to be independent of every other in terms of selection from a group. Should any variable, substituent, or term (e.g., aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(═O)N(R)— may be attached to the parent moiety at either the carbon or the nitrogen.

As used herein, the term “2-cyclylcyclopropan-1-amine compound” refers to a compound comprising a 2-cyclylcyclopropan-1-amine moiety or a pharmaceutically acceptable salt or solvate thereof. Exemplary 2-cyclylcyclopropan-1-amine compounds are, without limitation, 2-arylcyclopropan-1-amine compounds (such as 2-phenylcyclopropan-1-amine compounds) and 2-heteroarylcyclopropan-1-amine compounds (such as 2-pyridinylcyclopropan-1-amine compounds or 2-thiazolylcyclopropan-1-amine compounds).

As used herein, the term “2-arylcyclopropan-1-amine compound” refers to a compound comprising a 2-arylcyclopropan-1-amine moiety or a pharmaceutically acceptable salt or solvate thereof.

As used herein, the term “2-heteroarylcyclopropan-1-amine compound” refers to a compound comprising a 2-heteroarylcyclopropan-1-amine moiety or a pharmaceutically acceptable salt or solvate thereof.

As used herein, the term “2-phenylcyclopropan-1-amine compound” refers to a compound comprising a 2-phenylcyclopropan-1-amine moiety or a pharmaceutically acceptable salt or solvate thereof.

As used herein, the term “2-pyridinylcyclopropan-1-amine compound” refers to a compound comprising a 2-pyridinylcyclopropan-1-amine moiety or a pharmaceutically acceptable salt or solvate thereof.

As used herein, the term “2-thiazolylcyclopropan-1-amine compound” refers to a compound comprising a 2-thiazolylcyclopropan-1-amine moiety or a pharmaceutically acceptable salt or solvate thereof.

As used herein, the term “phenelzine compound” refers to a compound comprising a 2-phenylethylhydrazine moiety or a pharmaceutically acceptable salt or solvate thereof.

As used herein, the term “propargylamine compound” refers to a compound comprising a propargylamine moiety or a pharmaceutically acceptable salt or solvate thereof. An exemplary propargylamine compound is, without limitation, pargyline (N-benzyl-N-methylprop-2-yn-1-amine).

In reference to the substituents referred to above, as the skilled artisan is aware, the appropriate selection of the substituents can be made in view of the disclosure herein to provide LSD1 inhibitors, selective LSD1 inhibitors, and dual LSD1/MAO-B inhibitors for use in the methods and compositions of the invention.

Preferably, the LSD1 inhibitor for use in the invention is a selective LSD1 inhibitor or dual inhibitor of LSD1 and MAO-B. In one preferred aspect, the selective LSD1 or dual LSD1/MAO-B inhibitor has a molecular weight of less than 700 Daltons. In one preferred aspect, the selective LSD1 or dual LSD1 MAO-B inhibitor has a molecular weight of less than 500 Daltons. In one preferred aspect, the selective LSD1 or dual LSD1 MAO-B inhibitor has a molecular weight of less than 300 Daltons.

Preferably, the LSD1 inhibitor comprises five or less amide bonds (—NH—CO—). Preferably, the LSD1 inhibitor comprises three or less amide bonds (—NH—CO—).

In one aspect, the LSD1 inhibitor for use in the invention has zero amide bonds.

In one aspect, the selective LSD1 inhibitors and dual LSD1/MAOB inhibitors for use in the invention desirably inhibit LSD1 and/or MAOB selectively compared to MAOA, thus avoiding deleterious side effects associated with administration to animals, including humans, of MAOA inhibitors. As the inventors have described herein, the selective LSD1 inhibitors and the dual LSD1/MAOB inhibitors can be administered in a such a way to an individual e.g., a mammal or human, to achieve concentration in vivo that are expected to inhibit LSD1 and/or MAO-B while avoiding the toxicity associated with inhibition of MAOA and these concentrations are sufficient enough to improve symptoms associated with inflammation or inflammatory diseases or conditions.

The invention provides a pharmaceutical composition for treating inflammation or inflammatory diseases or conditions comprising a pharmaceutically acceptable carrier and a compound which is an inhibitor of LSD1. Preferably the LSD1 inhibitor is a selective LSD1 inhibitor or a dual LSD1/MAOB inhibitor. The ability of a compound to inhibit LSD1 and/or MAOB and its IC50 values for LSD1, MAO-A and MAO-B can be determined in accordance with the experimental protocol described in Example 1. In one specific embodiment, LSD1 inhibitors for use in the invention are as defined above and are chosen from a phenylcyclopropylamine derivative or analog, a phenelzine derivative or analog, or a propargylamine derivative or analog. In another embodiment, the LSD1 inhibitor for use in the invention is chosen from a 2-cyclylcyclopropan-1-amine compound, a phenelzine compound and a propargylamine compound; more preferably, the LSD1 inhibitor for use in the invention is a 2-cyclylcyclopropan-1-amine compound, preferably a 2-arylcyclopropan-1-amine compound or a 2-heteroarylcyclopropan-1-amine compound, and still more preferably a 2-phenylcyclopropan-1-amine compound, a 2-pyridinylcyclopropan-1-amine compound or a 2-thiazolylcyclopropan-1-amine compound.

The invention provides a pharmaceutical composition for treating inflammation or inflammatory diseases or conditions comprising a pharmaceutically acceptable carrier and a compound which is a selective inhibitor of LSD1. Preferably, LSD1 selective inhibitors (or selective LSD1 inhibitors) have IC50 values for LSD1 which are at least two-fold lower than the IC50 value for MAO-A and/or MAO-B. Even more preferably, LSD1 selective inhibitors have IC50 values for LSD1, which are at least five-fold lower than the IC50 value for MAO-A and/or MAO-B. Yet even more preferably, LSD1 selective inhibitors have IC50 values for LSD1 which are at least ten-fold lower than the IC50 value for MAO-A and/or MAO-B. The ability of a compound to inhibit LSD1 and its IC50 values for LSD1, MAO-A and MAO-B can be determined in accordance with the experimental protocol described in Example 1. In one specific embodiment, a selective LSD1 inhibitors for use in the invention are as defined above and are chosen from a phenylcyclopropylamine derivative or analog, a phenelzine derivative or analog, or a propargylamine derivative or analog. In another embodiment, the selective LSD1 inhibitor for use in the invention is chosen from a 2-cyclylcyclopropan-1-amine compound, a phenelzine compound and a propargylamine compound; more preferably, the selective LSD1 inhibitor for use in the invention is a 2-cyclylcyclopropan-1-amine compound, preferably a 2-arylcyclopropan-1-amine compound or a 2-heteroarylcyclopropan-1-amine compound; and still more preferably a 2-phenylcyclopropan-1-amine compound, a 2-pyridinylcyclopropan-1-amine compound or a 2-thiazolylcyclopropan-1-amine compound.

The invention also provides a pharmaceutical composition comprising a pharmaceutically acceptable carrier and a compound which is a dual inhibitor selective for LSD1 and MAO-B. Preferably, dual LSD1/MAO-B inhibitors have IC50 values for LSD1 and MAO-B which are at least two-fold lower than the IC50 value for MAO-A. Even more preferably, dual LSD1/MAO-B inhibitors have IC50 values for LSD1 and MAO-B which are at least five-fold lower than the IC50 value for MAO-A. Yet even more preferably, dual LSD1/MAO-B inhibitors have IC50 values for LSD1 and MAO-B which are at least ten-fold lower than the IC50 value for MAO-A. The ability of a compound to inhibit LSD1 and its IC50 values for LSD1, MAO-A and MAO-B can be determined in accordance with the experimental protocol described in Example 1. In one specific embodiment, dual selective LSD1/MAO-B inhibitors for use in the invention are as defined above and are chosen from a phenylcyclopropylamine derivative or analog, a phenelzine derivative or analog, or a propargylamine derivative or analog. In another embodiment, the selective LSD1 inhibitor for use in the invention is chosen from a 2-cyclylcyclopropan-1-amine compound, a phenelzine compound and a propargylamine compound; more preferably, the selective LSD1 inhibitor for use in the invention is a 2-cyclylcyclopropan-1-amine compound, preferably a 2-arylcyclopropan-1-amine compound or a 2-heteroarylcyclopropan-1-amine compound, and still more preferably a 2-phenylcyclopropan-1-amine compound, a 2-pyridinylcyclopropan-1-amine compound or a 2-thiazolylcyclopropan-1-amine compound.

Typically, compounds for use as LSD1 inhibitors, selective LSD1 inhibitors or dual inhibitors of LSD1 and MAO-B can be effective at an amount of from about 0.01 μg/kg to about 100 mg/kg per day based on total body weight. The active ingredient may be administered at once, or may be divided into a number of smaller doses to be administered at predetermined intervals of time. The suitable dosage unit for humans for each administration can be, e.g., from about 1 μg to about 2000 mg, preferably from about 5 μg to about 1000 mg, and even more preferably from about 0.01 mg to about 500 mg (e.g., from about 0.5 mg to about 500 mg). The active ingredient can be administered orally or by other routes of administration, e.g., IP, IV, etc. Preferably, the inhibitor is formulated and delivered in such a way as to achieve concentration in vivo that modulate the target activity, e.g., LSD1 and/or MAOB. Thus, in a specific embodiment, the effective amount of compound ranges from 0.05 μg/kg to about 100 mg/kg per day, preferably from 0.05 μg/kg to about 50 mg/kg.

It should be understood that the dosage ranges set forth above are exemplary only and are not intended to limit the scope of this invention unless specified. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. For example, useful components include sodium chloride, acetates, citrates or phosphates buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include skin, nasal, buccal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown et al., Ann. Rev. Med. 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by cross-linking high molecular weight biocompatible polymers into a network, which swells in water to form a gel like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci., 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm. 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art. Other pharmaceutically acceptable prodrugs of the compounds of this invention include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active ingredient can be formulated as a pharmaceutically acceptable salt. A “pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfates, phosphates, monohydrophosphates, dihydrophosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycoilates, tartrates, methane-sulfonates, propanesulfonates, naphthalene-1-sulfonates, naphthalene-2-sulfonates, or mandelates.

As used herein, a “pharmaceutically acceptable carrier” refers to a non-API (API refers to Active Pharmaceutical Ingredient) substances such as disintegrators, binders, fillers, and lubricants used in formulating pharmaceutical products. They are generally safe for administering to humans according to established governmental standards, including those promulgated by the United States Food and Drug Administration and the European Medical Agency.

The active compounds can also be administered in combination with another active agent that synergistically treats or prevents the same symptoms or is effective for another disease or symptom in the patient treated so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

As used herein, the term “anti-platelet agent” refers to any drug that decrease activation, aggregation, and/or adhesion of platelets, and inhibit thrombus formation. They are effective in the arterial circulation and they are widely used in primary and secondary prevention of thrombotic cerebrovascular or cardiovascular disease. The term “anti-platelet” encompasses a variety of commercially available anti-platelet drugs, including, but not limited to, Aspirin, Clopidogrel, Prasugrel, Ticlopidine, Cilostazol, Abciximab, Eptifibatide, Tirofiban, Dipyridamole or Epoprostenol.

As used herein, the term “anticoagulant agent” refers to any drug that inhibits or prevents blood coagulation. The term “anticoagulant” encompasses a variety of commercially available anticoagulat drugs, including, but not limited to, Heparin, Warfarin, low molecular weight Heparins, acenocoumarol, phenprocoumon or direct thrombin inhibitors.

As used herein, the term “anti-inflamatory agent” refers to any drug that inhibits or reduces inflammation. The term “anti-inflamatory” encompasses a variety of commercially available anti-inflamatory drugs, including, but not limited to, steroids, Salicylates (aspirin, diflunisal, salsalate), propionic acid derivatives (e.g., ibuprofen, naproxen, fenoprofen, ketoprofen, flurbiprofen, oxaprozin or loxoprofen), acetic acid derivatives (e.g., indomethacin, sulindac, etodolac, ketorolac, diclofenac or nabumetone), enolic acid derivatives (e.g., piroxicam, meloxicam, tenoxicam, droxicam, lornoxicam, isoxicam), fenamic acid derivatives (e.g., mefenamic acid, meclofenamic acid, flufenamic acid or tolfenamic acid), selective COX-2 inhibitors (e.g., celecoxib, rofecoxib, valdecoxib, parecoxib, lumiracoxib, etoricoxib, firocoxib) sulphonanilides (nimesulide) and licofelone.

As used herein, the term “individual in need of treatment” encompasses individuals who have symptoms of inflammation or an inflammatory disease or conditions, those who have been diagnosed with inflammation, an inflammatory disease or condition or a related disease or condition.

The examples described herein are intended to illustrate different aspects of the invention by exemplification and are not intended to limit the scope of the claims or invention.

### EXAMPLES

### Example 1

### Biochemical Assays

Compounds for use in the methods of the invention can be identified by their ability to inhibit LSD1. The ability of compounds to inhibit LSD1 can be tested as follows. Human recombinant LSD1 protein was purchased from BPS Bioscience Inc. In order to monitor LSD1 enzymatic activity and/or its inhibition rate by the LSD1 inhibitor(s) of interest, di-methylated H3-K4 peptide (Millipore) was chosen as a substrate. The demethylase activity was estimated, under aerobic conditions, by measuring the release of H₂O₂ produced during the catalytic process, using the Amplex® Red peroxideperoxidase-coupled assay kit (Invitrogen).

Briefly, a fixed amount of LSD1 was incubated on ice for 15 minutes, in the absence and/or in the presence of various concentrations of inhibitor (e.g., from 0 to 75 μM, depending on the inhibitor strength). Tranylcypromine (Biomol International) was used as a control for inhibition. Within the experiment, each concentration of inhibitor was tested in duplicate. After leaving the enzyme interacting with the inhibitor, 12.5 μM of di-methylated H3-K4 peptide was added to each reaction and the experiment was left for 1 hour at 37° C. in the dark. The enzymatic reactions were set up in a 50 mM sodium phosphate, pH 7.4 buffer. At the end of the incubation, Amplex® Red reagent and horseradish peroxidase (HPR) solution were added to the reaction according to the recommendations provided by the supplier (Invitrogen), and left to incubate for 30 extra minutes at room temperature in the dark. A 1 μM H₂O₂ solution was used as a control of the kit efficiency. The conversion of the Amplex® Red reagent to resorufin due to the presence of H₂O₂ in the assay, was monitored by fluorescence (excitation at 540 nm, emission at 590 nm) using a microplate reader (infinite 200, Tecan). Arbitrary units were used to measure level of H₂O₂ produced in the absence and/or in the presence of inhibitor.

The maximum demethylase activity of LSD1 was obtained in the absence of inhibitor and corrected for background fluorescence in the absence of LSD1. The Ki (IC50) of each inhibitor was estimated at half of the maximum activity.

Human recombinant monoamine oxidase proteins MAO-A and MAO-B were purchased from Sigma Aldrich. MAOs catalyze the oxidative deamination of primary, secondary and tertiary amines. In order to monitor MAO enzymatic activities and/or their inhibition rate by inhibitor(s) of interest, a fluorescent-based (inhibitor)-screening assay was set up. 3-(2-Aminophenyl)-3-oxopropanamine (kynuramine dihydrobromide, Sigma Aldrich), a non fluorescent compound was chosen as a substrate. Kynuramine is a non-specific substrate for both MAOs activities. While undergoing oxidative deamination by MAO activities, kynuramine is converted into 4-hydroxyquinoline (4-HQ), a resulting fluorescent product.

The monoamine oxidase activity was estimated by measuring the conversion of kynuramine into 4-hydroxyquinoline. Assays were conducted in 96-well black plates with clear bottom (Corning) in a final volume of 100 μL. The assay buffer was 100 mM HEPES, pH 7.5. Each experiment was performed in duplicate within the same experiment.

Briefly, a fixed amount of MAO (0.25 μg for MAO-A and 0.5 μg for MAO-B) was incubated on ice for 15 minutes in the reaction buffer, in the absence and/or in the presence of various concentrations of inhibitor (e.g., from 0 to 50 μM, depending on the inhibitor strength). Tranylcypromine (Biomol International) was used as a control for inhibition.

After leaving the enzyme(s) interacting with the inhibitor, 60 to 90 μM of kynuramine was added to each reaction for MAO-B and MAO-A assay respectively, and the reaction was left for one hour at 37° C. in the dark. The oxidative deamination of the substrate was stopped by adding 50 μL (v/v) of NaOH 2N. The conversion of kynuramine to 4-hydroxyquinoline, was monitored by fluorescence (excitation at 320 nm, emission at 360 nm) using a microplate reader (Infinite 200, Tecan). Arbitrary units were used to measure levels of fluorescence produced in the absence and/or in the presence of inhibitor.

The maximum of oxidative deamination activity was obtained by measuring the amount of 4-hydroxyquinoline formed from kynuramine deamination in the absence of inhibitor and corrected for background fluorescence in the absence of MAO enzymes. The Ki (IC50) of each inhibitor was determined at Vmax/2.

### Example 2

### LSD1 and LSD1/MAO-B Dual Inhibitors

TABLE 1
Exemplary IC50 values for selected compounds against LSD1,
MAO-A, and MAO-B, obtained using the assays of Example 1.

| | LSD1 IC50 | MAO-A IC50 | MAO-B IC50 |
|---|---|---|---|
| Compound No. | (uM) | (uM) | (uM) |
| Compound 1 | <0.20 | >2 | <0.20 |
| Compound 2 | <0.20 | >2 | <0.20 |
| Compound 3 | 0.10 | >2 | >2 |
| Compound 4 | <0.10 | >2 | >2 |
| Compound 6 | <0.20 | >1 | >0.5 |
| Compound 7 | <0.07 | >0.2 | >1 |
| Compound 8 | <0.07 | >2 | >2 |
| Compound 9 | <0.07 | >1 | >10 |

Compounds 1-4 and 6-9 are cyclylcyclopropylamine derivatives or analogs as described in WO2010/043721 (PCT/EP2009/063685), WO2010/084160 (PCT/EP2010/050697), WO2011/035941 (PCT/EP2010/055131), WO2011/042217 (PCT/EP2010/055103), WO2012/013727 and EP applications number EP10171345, EP10187039 andEP10171342.

Compound 1 corresponds to

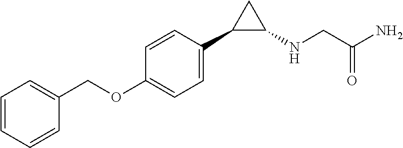 [SMILES: NC(=O)CN[C@H]1C[C@@H]1c1ccc(OCc2ccccc2)cc1]

and can be prepared as disclosed in WO 2011042217.

Compound 2 corresponds to the (−)-isomer of compound 1 (i.e. the enantiomer having a negative optical rotation), and can be prepared following the methods disclosed in WO 2011042217.

Compound 3 is

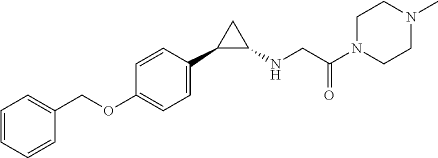 [SMILES: CN1CCN(C(=O)CN[C@H]2C[C@@H]2c2ccc(OCc3ccccc3)cc2)CC1]

and can be prepared as disclosed in WO 2010043721.

Compound 4 is

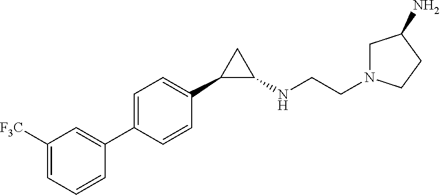 [SMILES: N[C@H]1CCN(CCN[C@H]2C[C@@H]2c2ccc(-c3cccc(C(F)(F)F)c3)cc2)C1]

and can be prepared as disclosed in WO 2011035941.

Compound 6 is

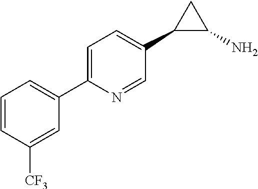 [SMILES: N[C@H]1C[C@@H]1c1ccc(-c2cccc(C(F)(F)F)c2)nc1]

and can be prepared as disclosed in WO 2012013727.

Compound 7 is

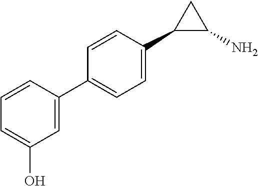 [SMILES: N[C@H]1C[C@@H]1c1ccc(-c2cccc(O)c2)cc1]

and can be prepared as disclosed in WO 2012013727.

Compound 8 is

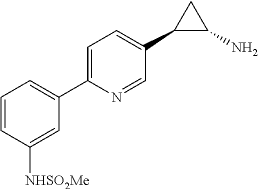

and can be prepared as disclosed in WO 2012013727.

Compound 9 is

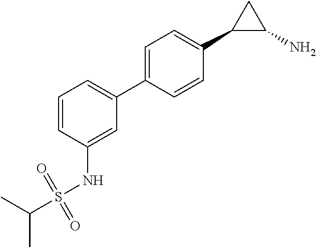 [SMILES: CC(C)S(=O)(=O)Nc1cccc(-c2ccc([C@H]3C[C@@H]3N)cc2)c1]

and can be prepared as disclosed in WO 2012013727.

The stereochemistry shown in the chemical structures depicted above for compounds 1 and 3 to 9 is only intended to show that the compounds have the “trans” configuration in respect to the substituents on the cyclopropyl ring, it does not refer to absolute stereochemistry. Compounds 1 and 3 to 9 are “trans” racemic mixtures, while compound 2 is a single stereoisomer.

The IC50 value of compound 3 for LSD1 was initially determined to be <0.10 μM, the IC50 values of compound 6 were initially determined to be >0.5 μM for MAO-A and >1 μM for MAO-B, and the IC50 value of compound 7 for MAO-A was initially determined to be >1 μM. In a further, more elaborate determination, the IC50 values indicated in Table 1 have been obtained. These further values confirm that compounds 1 and 2 are dual LSD1/MAO-B selective inhibitors and compounds 3 to 9 are selective LSD1 inhibitors.

### Example 3

### LSD1 and LSD1/MAO-B Dual Inhibitors Increase Histone Lysine Methylation in Cell-Based Assays

Histone from SH-SY5Y cells grown in the presence of Compound Dual-1 (a dual LSD1/MAOB inhibitor) (designated as Compound 1 in Example 2 above) or tranylcypromine (Parnate™) for one, two, and three days were extracted and subjected to western blot analysis using a commercially available antibody specific for dimethylated H3K4. B-actin was used as a loading control.

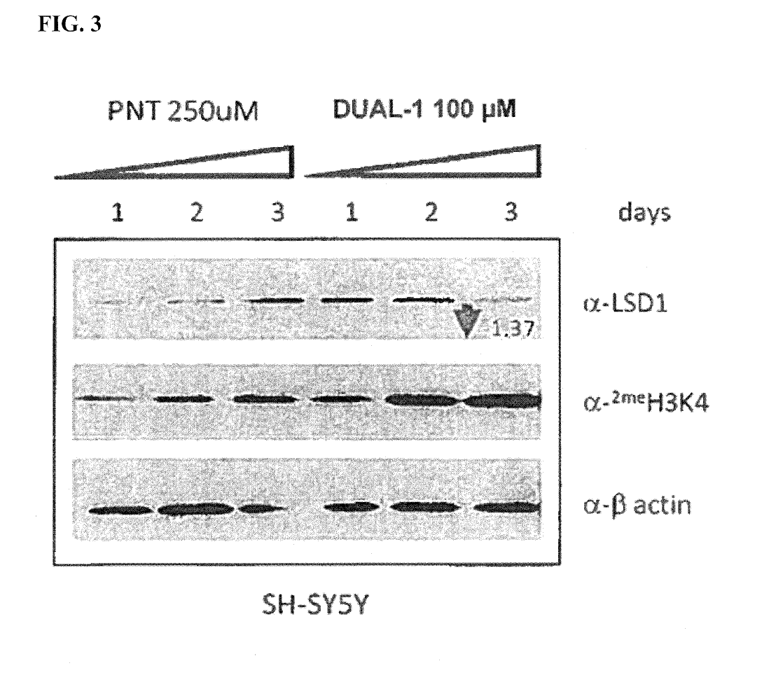

The results of a western blot stained for H3K4 methylation with SH-SY5Y cells grown in the presence of Compound Dual-1 or tranylcypromine (parnate) for one, two, and three days are shown in FIG. 3 and indicate that this compound, Dual-1, increases H3K4 methylation in cells in a time dependent manner and furthermore Compound Dual-1 appears to be ten-fold or more potent at increasing global demethylated H3K4 levels as compared to tranylcypromine.

Furthermore, the inventors have conducted similar studies for other dual inhibitors of LSD1/MAOB and with selective LSD1 inhibitors and found that these compounds can increase demethylated H3K4 levels in similarly performed assays.

### Example 4

### LSD1 Inhibitors can be Administered Safely to Mammals

Maximum tolerated dose studies and pharmacokinetics for several LSD1 inhibitors were assessed to determine if the compound can be administered to mammals safely at doses that are expected to achieve therapeutic effects. Results in chronic dosing experiments indicate that therapeutic levels can be reached in vivo.

### Example 5

### LSD1 Inhibitors Reduce Platelet Levels in Mammals

Method for Determination of Effects of LSD1 Inhibitors on Platelets:

Three mice were treated for five consecutive days with the compounds and doses indicated in Table 2. On the fifth day, 60 minutes after the administration, mice were sacrificed and blood was collected in sodium citrate-containing tubes for hemogram analysis. Platelet levels were determined and referred as % of platelets compared with the levels found in mice treated with vehicle. Platelet levels were determined in a standard hematology analyzer (Abacus Junior Vet, from Diatron) following the manufacturer's instructions.

20% 2-hydroxypropyl-P-cyclodextrin in H₂O was used as a vehicle. When necessary, 10% DMSO was also added in the vehicle. Each day, compounds were administered in a single intraperitoneal injection with administration volumes of 15 ml/kg.

Mice strain was Hsd:Athymic Nude-Foxn1nu. Animals were maintained in air and temperature controlled cages with regular supply of water and food.

TABLE 2
Results of platelet levels after five consecutive once daily
injections of LSD1 inhibitors at the indicated dose.

| | Dose | % platelets vs. |
|---|---|---|
| Compound | (mg/kg) | Vehicle |
| Compound 1 | 5 | 91 |
| | 10 | 66 |
| | 20 | 55 |
| | 40 | 34 |
| Compound 2 | 20 | 35 |
| | 40 | 27 |
| | 60 | 16 |
| Compound 3 | 1 | 46 |
| | 3 | 16 |
| | 10 | 5 |
| Compound 4 | 3 | 87 |
| | 9 | 30 |
| | 10 | 19 |
| | 18 | 19 |
| | 30 | 7 |
| | 36 | 11 |
| Compound 6 | 10 | 87 |
| | 30 | 63 |
| Compound 7 | 17 | 56 |
| | 34 | 46 |
| | 52 | 63 |
| Compound 8 | 20 | 68 |
| | 40 | 44 |
| Compound 9 | 30 | 10 |

Compounds 1-4 and 6-9 in Table 2 are the same compounds 1-4 and 6-9 as described in Example 2.

These results show that LSD1 inhibitors, including selective LSD1 inhibitors and dual inhibitors of LSD1 and MAOB, reduce platelet levels in vivo. The effect on platelet reduction is reversible and quickly reverts after interruption of treatment. As a result of their platelet-reducing activity, LSD1 inhibitors, including in particular the specific LSD1 inhibitors disclosed and described herein, are useful in the treatment or prevention of inflammation or an inflammatory disease or condition.

These inhibitors can also reduce the levels of other blood cells, as shown below for compound 3:

| | | Dose (mg/kg) | | |
|---|---|---|---|---|
| | Vehicle | 1 | 3 | 10 |
| White Blood Cells | 100 | 86 | 76 | 29 |
| Lymphocytes | 100 | 115 | 90 | 35 |
| Granulocytes | 100 | 69 | 63 | 23 |
| Red blood cells | 100 | 103 | 101 | 92 |

Measurements of all blood cell types were conducted in the same manner as described above for platelets. Data in the table above are expressed as the % of cells vs vehicle.

Various types of white blood cells (WBC), including granulocytes and lymphocytes, are involved in the initiation and maintenance of inflammation. Also red blood cells (RBC) have been reported to be involved in inflammatory processes; Kiefmann et al (Blood 2008, 111(10) 5205-14) reported that in hypoxic lungs RBC release reactive oxygen species (ROS), activate endothelium and trigger leukocyte recruitment, thus contributing to hypoxia-induced inflammation. Given the involvement of WBC and RBC in inflammation, the effect of LSD1 inhibitors on such blood cells in addition to platelets further supports the use of LSD1 inhibitors for the treatment of inflammation and inflammatory diseases or conditions.

1-4. (canceled)

5. A method of treating or preventing inflammation or an inflammatory disease or condition, comprising administering to an individual a therapeutically effective amount of a LSD1 inhibitor.

6. A method of treating or preventing a symptom of inflammation or an inflammatory disease or condition, comprising administering to an individual a therapeutically effective amount of a LSD1 inhibitor.

7. The method of claim 5 wherein said inflammation or inflammatory disease or condition is selected from atherosclerosis, a respiratory inflammatory disorder, respiratory distress syndrome, asthma, COPD, bronchial hyperresponsiveness, bronchoconstriction, airway inflammation, airway remodeling, cystic fibrosis, chronic inflammatory bowel disease, ulcerative colitis, Crohn's disease, a chronic skin inflammatory disease, psoriasis, atopic dermatitis, mesangial glomerulonephritis, Kawasaki disease, disseminated intravascular inflammation, Caffey disease, TRAP syndrome, allergic vasculitis, arthritis, vasculitis, coronary artery disease, carotid artery disease, transplant vasculopathy, rheumatoid arthritis, hepatic cirrhosis, and nephritis.

8-12. (canceled)

13. The method of claim 5 wherein said LSD1 inhibitor is a small molecule inhibitor of LSD1.

14. The method of claim 5 wherein said LSD1 inhibitor is a selective LSD1 inhibitor.

15. (canceled)

16. The method of claim 5 wherein said LSD1 inhibitor is an irreversible or a reversible amine oxidase inhibitor.

17. The method of claim 5 wherein said LSD1 inhibitor is a 2-cyclylcyclopropan-1-amine compound, a phenelzine compound or a propargylamine compound.

18. The method of claim 5 wherein said LSD1 inhibitor is a 2-arylcyclopropan-1-amine compound or a 2-heteroarylcyclopropan-1-amine compound.

19-20. (canceled)

21. The method of claim 5 wherein said LSD1 inhibitor is a 2-cyclylcyclopropan-1-amine compound of formula (I) or an enantiomer, a diastereomer or a mixture of stereoisomers thereof, or a pharmaceutically acceptable salt or solvate thereof: 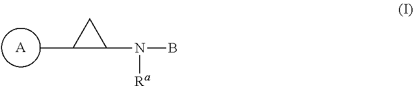
wherein:
A is cyclyl optionally having 1, 2, 3 or 4 substituents A′;
each A′ is independently selected from -L¹-cyclyl, alkyl, alkenyl, alkynyl, alkoxy, amino, amido, —CH₂—CO—NH₂, alkylamino, hydroxyl, nitro, halo, haloalkyl, haloalkoxy, cyano, sulfonyl, sulfinyl, sulfonamide, acyl, carboxyl, carbamate and urea, wherein the cyclyl moiety comprised in said -L¹-cyclyl is optionally further substituted with one or more groups independently selected from halo, haloalkyl, haloalkoxy, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cyano, sulfonyl, sulfinyl, sulfonamide, acyl, carboxyl, carbamate and urea;
each L¹ is independently selected from a covalent bond, —(CH₂)₁₋₆—, —(CH₂)₀₋₃—O—(CH₂)₀₋₃—, —(CH₂)₀₋₃—NH—(CH₂)₀₋₃— and —(CH₂)₀₋₃—S—(CH₂)₀₋₃—;
B is L²-cyclyl, —H, -L²-CO—NH₂, -L²-CO—NR¹R² or -L²-CO—R³, wherein the cyclyl moiety in said -L²-cyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate and urea;
R^{a} is —H or alkyl;
R¹ and R² are each independently selected from —H, alkyl, alkynyl, alkenyl, -L-carbocyclyl, -L-aryl, and -L-heterocyclyl, wherein said alkyl, said alkynyl or said alkenyl is optionally substituted with one or more groups independently selected from halo, haloalkoxy, haloaryl, aryl, arylalkoxy, aryloxy, alkoxy, amino, amido, alkylamino, hydroxyl, nitro, —CH₂—CO—NH₂, heteroaryl, heteroarylalkoxy, heteroaryloxy, cycloalkyl, cycloalkylalkoxy, cycloalkoxy, heterocycloalkyl, heterocycloalkylalkoxy, heterocycloalkoxy, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate and urea, and further wherein the carbocyclyl moiety in said -L-carbocyclyl, the aryl moiety in said -L-aryl, or the heterocyclyl moiety in said -L-heterocyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate and urea;
R³ is selected from -L-heterocyclyl, -L-carbocyclyl, -L-aryl, —H, and alkoxy, wherein the carbocyclyl moiety in said -L-carbocyclyl, the heterocyclyl moiety in said -L-heterocyclyl or the aryl moiety in said -L-aryl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate and urea;
each L is independently selected from —(CH₂)ₙ—(CH₂)ₙ—, —(CH₂)ₙC(═O)(CH₂)ₙ—, —(CH₂)ₙC(═O)NH(CH₂)ₙ—, —(CH₂)ₙNHC(═O)O(CH₂)ₙ—, —(CH₂)ₙNHC(═O)NH(CH₂)ₙ—, —(CH₂)ₙNHC(═S)S(CH₂)ₙ—, —(CH₂)ₙOC(═O)S(CH₂)ₙ—, —(CH₂)ₙNH(CH₂)ₙ—, —(CH₂)ₙO(CH₂)ₙ—, —(CH₂)ₙS(CH₂)ₙ—, and —(CH₂)ₙNHC(═S)NH(CH₂)ₙ—, wherein each n is independently selected from 0, 1, 2, 3, 4, 5, 6, 7, and 8; and
L² is C₁₋₁₂ alkylene which is optionally interrupted by one or more groups independently selected from —O—, —S—, —NH—, -(alkyl)-, —CO—, —CO—NH— and —CO—N(alkyl)-, or L² is a covalent bond.

22. The method of claim 21 wherein R^{a} is —H.

23. The method of claim 21 wherein A is aryl or heteroaryl and wherein A is unsubstituted or has 1 or 2 substituents A′.

24. (canceled)

25. The method of claim 23 wherein A is phenyl, pyridinyl, pyrimidinyl, thiophenyl, benzothiophenyl, pyrrolyl, indolyl, furanyl, or thiazolyl, and wherein A is unsubstituted or has 1 or 2 substituents A′.

26-34. (canceled)

35. The method of claim 21 wherein B is -L²-cyclyl, and further wherein the cyclyl moiety in said -L²-cyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate and urea.

36-37. (canceled)

38. The method of claim 35 wherein the cyclyl moiety in said -L²-cyclyl is aryl or cycloalkyl.

39. The method of claim 35 wherein the cyclyl moiety in said -L²-cyclyl is heteroaryl or heterocycloalkyl.

40-42. (canceled)

43. The method of claim 35 wherein L² is —(CH₂)₁₋₄—, —CH₂—CO— or a covalent bond.

44-50. (canceled)

51. The method of claim 21, wherein B is —H.

52. The method of claim 21, wherein B is -L²-CO—NH₂, —(CH₂)₁₋₄—CO—NH₂, -L²-CO—NR¹R², —(CH₂)₁₋₄—CO—NR¹R², -L²-CO—R³ or —(CH₂)₁₋₄—CO—R³.

53-60. (canceled)

61. The method of claim 21 wherein the substituents on the cyclopropane ring are in trans configuration.

62-68. (canceled)

69. The method of claim 5, wherein said LSD1 inhibitor is to be administered in combination with an anti-inflammatory agent.

70-71. (canceled)
